(19) United States
(12) Patent Application Publication
(10) Pub. No.: US 2026/0174645 A1
(43) Pub. Date: Jun. 25, 2026

(54) CROP NUTRITION COMPOSITION

(71) Applicant: Komal BHUKHANWALA, Mumbai (IN)

(72) Inventor: Harsha RAMANAND NAIK, Thane (IN)

(21) Appl. No.: 19/128,834

(22) PCT Filed: Jan. 7, 2023
(86) PCT No.: PCT/IB2023/050129

§ 371 Date: May 9, 2025

(30) Foreign Application Priority Data
Nov. 18, 2022 (IN) 202221066608

(51) Int. Cl. A61K 8/27

(52) U.S. Cl. CPC A61K 8/27

(57) ABSTRACT

The present invention relates to a crop nutrition composition in the form of liquid suspension comprising a homogeneous mixture of one or more of water insoluble Magnesium salt, complex or derivative thereof in the range of 1-70% w/w of the total composition and one or more of water insoluble Zinc salt, complex or derivative thereof in the range of 1-50% w/w of the total composition and one or more of water insoluble Iron salt, complex or derivative thereof in the range of 1-50% w/w of the total composition with at least one agrochemically acceptable excipient; wherein elemental Iron is present in the range of 0.01% to 40% w/w of the total composition and wherein elemental Zinc is present in the range of 0.01% to 40% w/w of the total composition and wherein elemental Magnesium is present in the range of 0.01% to 50% w/w of the total composition and wherein the composition comprises of particles in the size range of 0.1 micron-20 microns. The liquid suspension composition is in the form of a suspension concentrate, oil dispersion or a suspo-emulsion. The present invention further relates to a method of treating plants and meeting their nutritional requirement by making essential nutrients like Magnesium, Zinc and Iron available to them and also unlocking other micronutrients and trace elements present in the soil which hitherto were not available because of various factors primarily being soil degradation on account of excessive use of synthetic fertilizers. The present invention also relates to strengthening the plants so as to withstand pest infestation. The present invention also relates to a method of biofortification of plant with essential micronutrients.

### FIELD OF THE INVENTION

The present invention relates to a crop nutrition composition in the form of liquid suspension comprising a homogeneous mixture of an effective amount of one or more of water insoluble Magnesium salt, complex or derivative thereof and one or more of water insoluble Zinc salt, complex or derivative thereof and one or more of water insoluble Iron salt, complex or derivative thereof with at least one agrochemically acceptable excipient. The liquid suspension composition of the present invention comprises of particles in the size range of 0.1 micron to 20 microns. The liquid suspension composition is in the form of a suspension concentrate, an oil dispersion or a suspo-emulsion composition.

The invention further relates to a liquid suspension composition comprising a homogeneous mixture of one or more of water insoluble Magnesium salt, complex or derivative thereof in the range of 1%-70% w/w of the total composition and one or more of water insoluble Zinc salt, complex or derivative thereof in the range of 1%-50% w/w of the total composition and one or more of water insoluble Iron salt, complex or derivative thereof in the range of 1%-50% w/w of the total composition with at least one agrochemically acceptable excipient, wherein elemental Iron is present in the range of 0.01% to 40% w/w of the total composition and wherein elemental Zinc is present in the range of 0.01% to 40% w/w of the total composition and wherein elemental Magnesium is present in the range of 0.01% to 50% w/w of the total composition and wherein the composition comprises of particles in the size range of 0.1 micron to 20 microns. The liquid suspension composition is in the form of a suspension concentrate, an oil dispersion or a suspo-emulsion composition.

The present invention further relates to a method of treating plants and meeting their nutritional requirement by making essential nutrients like Magnesium, Zinc and Iron available to them and also unlocking other micronutrients and trace elements present in the soil which hitherto were not available because of various factors primarily being soil degradation on account of excessive use of synthetic fertilizers. The present invention also relates to strengthening the plants so as to withstand pest infestation.

The present invention also relates to a method of biofortification of plant with essential micronutrients.

### BACKGROUND OF THE INVENTION

In describing the embodiments of the invention, specific terminology is chosen for the sake of clarity. However, it is not intended that the invention be limited to the specific terms so selected and it is to be understood that each specific term includes all technical equivalents that operate in a similar manner to accomplish a similar purpose.

Nutrition is the key element in growth and development of crops. Poor and inadequate availability of nutrients to the plants results in lack of proper growth and physiological development. As a consequence, the plants become more susceptible to attack by pests. Other problems associated with agriculture are environmental conditions such as drought, biotic and abiotic stress, poor soil condition or depletion of nutrients in the soil which lead to reduction in the yield and quality of produce. Thus, providing adequate and balanced nutrition in a manner such that there is maximum uptake of nutrient by the plant, along with protection to the crops remains a great challenge. Optimizing the soil condition and managing the use of crop nutrients has been a long felt need of farmers to improve the nutrient use efficiency of crops. Significant research is being carried out so as to improve soil and plant health, provide better economic returns to farmers and also reduce the burden on the environment because of rampant use of synthetic pesticides.

In parallel, hidden hunger and micronutrient deficiencies across population in all major continents is rampant which contribute substantially to the global burden of diseases. Amongst the micronutrient deficiencies that people are normally suffering across the globe, Iron (Fe) and Zinc (Zn) are two important nutrients found in human nutrition and are amongst the most common micronutrient deficiencies in the world. Fe deficiency is seen in 20%-25% of the world population and Zn deficiency is seen in 17.3% of the world population (Cooper et al. 2012). One of the key underlying causes of this is the imbalanced fertilizer practice. Excessive and indiscriminate application of nutrients can cause severe imbalance and antagonism which results in nutrient deficient produce. It is thus a herculean and challenging task to grow food in quantity while maintaining quality in terms of nutrient content.

Further, modern agriculture is challenged by degraded soils on account of excessive use of synthetic fertilizers such as nitrogen, phosphatic and potash-based fertilizers, excessive cultivation, which in turn produce crops and harvest that are devoid of nutrients finally affecting human nutrition and health. More than 30% of the earth surface is covered by Calcareous soil which also pose a challenge in terms of providing adequate Zinc and Iron nutrients to the crop. Besides, the increasing labor & water shortage, demand for high and quality yields, current farming practices are greatly challenged by deteriorating soil health, depletion of water table, decreasing fertility of soil, leaching of fertilizers and pesticides, micronutrient deficiencies in the soil and so on. The use of excessive synthetic fertilizers has led to huge imbalance of soil nutrients. Almost more than double the amount of fertilizers such as Nitrogen, Phosphorous and Potassium have been applied nowadays to the soil than they were applied 20 or 30 years ago to achieve similar yields. It was observed that excessive nitrogen fertilizer leads to a reduction of exchangeable Calcium and Magnesium ions in the soil, making it unavailable to the plant which in turn results in retardation of plant growth and soil health. Also, the long-term use of synthetic NPK fertilizers make the soils acidic, degraded and also limits the uptake of other vital nutrients including Zinc, Iron, Calcium and Magnesium. The excessive amounts of Nitrogen, Phosphorous and Calcium, in the soil further lead to a nutrient imbalance and the final produce is devoid of essential nutrients, particularly Zinc and Iron (due to excessive Phosphorous) and Magnesium (due to excessive Calcium and Nitrogen).

The role of micronutrients as an essential element required for growth and reproduction by plants has been long known. Micronutrients plays an important role in balancing the crop nutrition. Further, it is also known that optimum levels of nutrients are required for the normal functioning, growth of the plants and any variance in the nutrient levels may cause hindrance in overall crop growth and cause its health to decline due to either a deficiency or toxicity. Poor availability of fertilizers or nutrients to the plants results in a lack of proper growth, resulting in the plants becoming more susceptible to attack by pests. In fact, it is also observed that even though there are some soil types that are carrying adequate amounts of micronutrients including Iron, Zinc and other elements, their bioavailability for crop uptake is limited due to various factors and the final harvest is deficient in these nutrients.

Besides the low concentration of essential micronutrients in soil, one of the root causes for the deficiency is the low availability of micronutrients in its oxidized form to plant roots. Further, leaching of water-soluble nutrients due to rain and irrigation also reduces the availability of nutrients in soil. Furthermore, managing nutrition of crops is difficult due to factors such as variable carbonate levels in soil, soil salinity, soil moisture, soil alkalinity, low temperature and concentration of other elements i.e. ‘competitive microelements’ which may also affect the availability of the micronutrients and at times lead to the deficiency of the micronutrients. Further, the ability of plants to respond to the availability of micronutrients ultimately affects human nutrition, both in terms of crop yield and the micronutrient concentration in the edible tissues. Therefore, proper nutrition is critical for optimizing the plant nutrition and metabolism, which in turn contributes to the overall crop yield and quality.

The interaction among plant nutrients can either be antagonistic or synergistic depending upon the mixture of elements and its composition, concentration etc. and that may influence nutrient use efficiency. Due to application of excess nutrients, plants may suffer from “nutrient antagonism” whereby an excess of a particular element may block the absorption of another element required by the plant and can happen with elements of a similar size and charge (positive or negative) which can result into deficiencies in the plant. Some of the most common antagonisms are iron blocking zinc, manganese (or the reverse), magnesium blocking calcium (or the reverse) and potassium blocking both magnesium and calcium. Another reason for a plant being deficient is “Binding” which occurs when elements mix together and bond, forming a compound that is insoluble and cannot be absorbed by plant's roots. Thus, it is imperative to apply balance amounts of the most limiting nutrients to obtain the highest yield while minimizing nutrient losses. One of the articles titled “Iron-magnesium antagonism in growth and metabolism of radish; Agarwala S. C, and S. C. Mehrotra et al; 1984” (reported the iron-magnesium antagonism in crops while another article titled “Effects of Nutrient Antagonism and Synergism on Yield and Fertilizer Use Efficiency; René P. J. J. Rietra, Marius Heinen et al; 2017” reported antagonism between Zinc and Magnesium. Further, antagonism between Fe and Zn is also well known (Alloway, 2008 & Kabata-Pendias, 2001).

Magnesium (Mg) is an essential macro element that is necessary for plant growth, health and development. Magnesium is involved in several different processes, including photosynthesis. The most important role of Magnesium is as a central atom or heart in the chlorophyll molecule. Without Magnesium, chlorophyll cannot capture Sun's energy required for photosynthesis. Magnesium also helps to activate specific enzyme system which are involved in a plant's normal metabolism. Furthermore, it is also needed for cell division and protein formation and is an essential component for plant respiration.

The availability of Magnesium in the soil depends on multiple factors. One of them being the source rock material, the degree of weathering, local climate and specific agricultural system, its management practices, such as crop type, cropping intensity, cropping rotation and fertilization practices. Due to its high mobility within the plant, Magnesium deficiency symptoms appear first on the lower and older leaves, before the symptoms become visible on the younger leaves. The symptoms show up as yellow leaves with green veins and around the edges (i.e. interveinal chlorosis). Purple, red or brown spots may also appear on the leaves. Magnesium and its importance in crop production and agriculture has been overlooked for some time, even though it is an essential element for plant growth and development. This is due to the fact that it is difficult to detect latent Magnesium deficiency.

Further, Iron (Fe) is also an essential nutrient element required for plant or crop growth, development and reproduction, however in relatively small amounts, thus making it a micronutrient. Iron is involved in many important physiological processes in plants such as the manufacturing process of chlorophyll and a range of enzymes and proteins. It also plays a vital role in respiration, nitrogen fixation, energy transfer and metabolism in crops and plants. Iron is relatively immobile ion and once incorporated into the tissues remain in the upper parts of the plants. As a result, the translocation of Iron from one plant part to another is restricted which leads to Iron deficiency. Such deficiency in plants or crops is commonly responsible for chlorosis (yellowing). Moreover, poor Iron nutrition also results in poor nodulation of legume crops, leading to reduced size and yield.

It was observed that managing Iron nutrition of crops is difficult due to factors such as carbonate levels in the soil, salinity, soil moisture, soil alkalinity, low temperature and concentration of other nutrient elements (e.g. competitive microelements such as Phosphorus, Calcium) which may also affect the Iron availability and at times leads to Iron deficiency. Also, the ability of plants to respond to Iron availability not only impacts the crop yield and the iron concentration in the edible plant tissues but ultimately affects the plant nutrition. Therefore, proper Iron assimilation by the crops is critical for optimizing crop nutrition and metabolism, which in turn contribute to the overall crop yield and quality.

The role of Zinc (Zn) as an essential micronutrient has also been long known. It is an important constituent of several enzymes, proteins that are responsible for driving many metabolic reactions in crops and also crucial to plant development. Zinc activates enzymes that are responsible for the synthesis of certain proteins. It is used in the formation of chlorophyll and some carbohydrates, conversion of starches to sugars and its presence in plant tissue helps the plant to withstand cold temperatures. Zinc is an essential element in the formation of auxins which help with growth regulation and stem elongation.

Zinc is immobile, due to which the deficiency symptoms occur in the new leaves. Typically, they are expressed as some varying pattern of chlorosis of the new leaves (often interveinal) and necrotic spots may form on the margins or leaf tips which results in formation of leaves which are smaller in size and often cupped upward or distorted. The symptoms also include poor bud development resulting in reduced flowering and branching, shorter internodes, giving a rosette appearance to the plant. Carbohydrate, protein, and chlorophyll formation is significantly reduced in Zinc-deficient plants. Therefore, a constant and continuous supply of Zinc is needed for optimum growth and maximum yield.

Though the benefits of micronutrients are well known, its deficiency has become widespread over the past several decades in most of the agricultural areas of the world, resulting in micronutrients being indicated as a limiting factor to improved plant growth, high yield and fertilizer efficiency.

Agricultural compositions which include micronutrient combinations are known in the art mostly in the form of powder or dust wherein the micronutrients are blended and mixed together. However, such powder-based compositions would lead to a non-uniform or non-homogeneous mixture of actives which may not be desirable in terms of its application and also poor uptake of the nutrition by the plants. Powder composition not only have issues with respect to practical application like generation of dust but also pose risk to the users mostly because of eye irritation, inhalation risk and skin irritation. Such formulations are also not easily dispersible and tend to clog the nozzles when applied via drip, making it unsuitable for use in irrigation system. Further, these compositions have also been found to have poor suspensibility which lead to random and non-uniform distribution of active ingredient on the target area which would cause undesirable effects and pose a problem in effective delivery of nutrients to the plant or crop and are also required to be used in large amounts.

Conventionally, micronutrient-based compositions are also known in the art in the form of bentonite granules or pastilles, pellets, granules prepared through molten process etc. Such products of micronutrient combinations in the form of granules or pellets or pastilles comprises of swelling clays and have been associated with several drawbacks. These compositions are generally bigger in size and include swelling clay which swell on contact with moisture and disintegrate into large particles of uneven size. Such granules or pastilles also lead to an irregular release of the micronutrients not meeting the plant nutritional requirement and eventually resulting in poor field efficacy. A gain, these types of micronutrient compositions are only suitable for broadcast applications, owing to their own disadvantages namely poor dispersion and suspensibility in water because of its disintegration into larger particle size, resulting in nozzle clogging in spray applications, posing a problem in delivery of nutrients to the plants or the crops. Due to these drawbacks, such prior art compositions containing micronutrients have negligible commercially viability or applicability in drip or sprinkler irrigation system which today is an essential mode of irrigation on account of labour shortage and water scarcity.

There are granular or powder compositions known in the art which involve the use of water-soluble nutrients. However, such compositions during heavy rainfall or irrigation tend to wash away and fail to be absorbed by the plants which in turn causes ground water contamination. As soils become more saline, plants are unable to draw as much water and nutrients from the soil. This results not only in a marked loss of efficiency but also has serious environmental consequence.

Compositions comprising fertilizer granules coated with micronutrient mixtures or water disintegrable granules of micronutrients are also known in the art. However, such compositions are designed in a manner such that they release the actives very slowly making the actives locked in the soil for prolonged period of time depriving the plant of their immediate nutritional requirement. As a consequence of the nutritional deficiency in the plants during their infancy, it makes them susceptible to various diseases eventually stunting their growth and yield. Further, water disintegrable granular compositions owing to non-uniform disintegration and distribution of particles suffer from their own set of drawbacks. On account of disintegration into random and non-uniform particles sizes, such compositions tend to clog the nozzles when applied via drip, making it unsuitable for use in modern day irrigation system.

No suitable liquid composition comprising Magnesium in combination with Iron and Zinc is known, which would make them available to the plant in effective quantities thus meeting the balance nutritional requirement of plants and address the drawbacks like nutrient antagonism of such compositions known in the art.

Despite the known antagonism between Zn—Fe, Zn—Mg and Mg—Zn, it has always been challenging to develop an agricultural composition that overcome this problem and successfully meet the nutritional requirement of plants. The present inventors surprisingly found that the composition of the present invention comprising Magnesium, Zinc and Iron was not only effective in overcoming the antagonism amongst these individual nutrients but also exhibited synergistic effect. It was found that the composition of the present invention when formulated at a specific particle size made the nutrients Magnesium, Zinc and Iron readily available for uptake by the plants. It was also noted by the present inventors that the application of the composition renders a greater and balanced uptake of not only Magnesium, Iron and Zinc but of other nutrients that remained entrapped in the soil and provide a natural bio-fortification solution in a sustainable manner, even in degraded soils.

It was observed that the surprising effect was noted when the present composition comprising a combination of water insoluble salts, complex or derivatives of Magnesium, Zinc and Iron in specific proportions was formulated into a liquid suspension along with a specific particle size distribution. The composition of the present invention was found to address the challenges of nutrient antagonism namely between Zinc and Iron, Magnesium and Zinc, Magnesium and Iron, etc. The present composition was further observed to prevent the leaching of these nutrients and make them available to the fullest extent for the uptake by crops and increase the overall yield.

It is known in the art that optimum absorption of majority of micronutrients or macronutrients by plants occur in soils at acidic or neutral pH. However, the inventors of the present invention surprisingly discovered that the composition of the present invention provided uptake of nutrients even in soils with alkaline pH or calcareous soils. So, in addition to overcome the challenge of nutrient antagonism, the composition of the present invention was found to be effective on all soil types making it a very viable composition for all geography. Further, it was noted that the presence of Magnesium (Mg) along with Zinc and Iron in the form of the composition of the present invention facilitated not only an uptake of significant proportion of Iron (Fe) and Zinc (Zn) present in the composition but also enabled plant uptake of micro nutrients like Boron (B), Manganese (Mn), Calcium (Ca) etc entrapped in the soil.

The composition of the present invention was found to play a vital role in regulating soil pH and facilitating the uptake of nutrients even in soils which have been degraded or whose pH have been altered because of excessive use of synthetic fertilizers. The composition of the present invention met the nutritional need of plants by providing a balanced uptake of essential nutrients like Zinc, Iron and Magnesium, thus overcoming the challenge of providing a nutrient rich crop in calcareous soils which is known to provide an antagonism challenge for the uptake of these nutrients especially Iron, Zinc and Magnesium (Singh et.al., 1990, 1993). It was further surprising to observe that the balanced uptake of nutrients leads to a healthier plant that could withstand pest infestation, a higher nutrient harvest in all soils types and finally improving the overall soil health. The present composition acts as a nutrient use efficient composition while meeting the need of crops by providing a multi nutritive solution with improved uptake by crops in a single application.

The inventors of the present application have determined that the crop nutrition composition in the form of liquid suspension comprising a homogeneous mixture of an effective amount of one or more of water insoluble Magnesium salt, complex or derivative thereof and an effective amount of one or more of water insoluble Zinc salt, complex or derivative thereof and an effective amount of one or more of water insoluble Iron salt, complex or derivative thereof with at least one agrochemically acceptable excipient wherein the composition comprises of particles in the size range of 0.1 micron to 20 microns demonstrate excellent field efficacy. The liquid suspension composition is in the form of suspension concentrate, an oil dispersion or a suspo-emulsion. The composition of the present invention further assists in regulating the soil pH so as to facilitate the balance uptake of micronutrients.

The composition of the present invention also exhibits superior physical characteristics such as suspensibility, viscosity, pourability and spontaneity of dispersion.

### SUMMARY OF THE INVENTION

The inventors have determined that a crop nutrition composition in the form of liquid suspension comprising a homogeneous mixture of an effective amount of one or more of water insoluble Magnesium salt, complex or derivative thereof and an effective amount of one or more of water insoluble Zinc salt, complex or derivative thereof and an effective amount of one or more of water insoluble Iron salt, complex or derivative thereof with at least one agrochemically acceptable excipient, provides the nutrients Magnesium, Zinc and Iron readily available for uptake by the plants and increase the overall yield in various crops and improves plant physiological parameters.

The liquid suspension composition of the present application comprises one or more water insoluble Iron salt, complex or derivative thereof in a concentration range of 1% to 50% by weight of the total composition, one or more water insoluble Magnesium salt, complex or derivative thereof in a concentration range of 1% to 70% by weight of the total composition, one or more water insoluble Zinc salt, complex or derivative thereof in a concentration range of 1% to 50% by weight of the total composition and at least one agrochemically acceptable excipient, wherein elemental Iron is present in the range of 0.01% to 40% w/w of the total composition and wherein elemental Zinc is present in the range of 0.01% to 40% w/w of the total composition and wherein elemental Magnesium is present in the range of 0.01% to 50% w/w of the total composition. The liquid suspension composition is in the form of a suspension concentrate, oil dispersion or suspo-emulsion.

Further, the liquid suspension composition comprises particles in the size range of 0.1 micron to 20 microns.

Furthermore, the invention relates to a process for preparing a crop nutrition composition in the form of liquid suspension comprising a homogeneous mixture of an effective amount of one or more of water insoluble Magnesium salt, complex or derivative thereof and an effective amount of one or more of water insoluble Zinc salt, complex or derivative thereof and an effective amount of one or more of water insoluble Iron salt, complex or derivative thereof with at least one agrochemically acceptable excipient.

The present invention further relates to a method of treating plants and meeting their nutritional requirement by making essential nutrients like Magnesium, Zinc and Iron available to them and also unlocking other micronutrients and trace elements present in the soil which hitherto were not available because of various factors primarily being soil degradation on account of excessive use of synthetic fertilizers. The present invention also relates to strengthening the plants so as to withstand pest infestation.

The present invention also relates to a method of bio-fortification of plant with essential micronutrients.

Further, the composition of present invention was found to be effective independent of the soil pH making it a viable composition for all types of soils. More importantly, it was noted that the presence of Magnesium along with Zinc and Iron in the form of composition of present invention facilitates not only the uptake of Iron and Zinc in the composition but also enabled the plant uptake of nutrients like Boron, Manganese, Calcium etc entrapped in the soil. The composition of the present invention was found to play a vital role in regulating soil pH and facilitating the uptake of nutrients even in soils which have been degraded or whose pH have been altered because of excessive use of synthetic fertilizers. It was further surprisingly observed that the composition of the present invention provides a balanced uptake of all nutrients including Zinc, Iron and Magnesium, thus overcoming the challenge of providing a nutrient rich crop in calcareous soils which is known to provide an antagonism challenge for the uptake of these nutrients. It was further surprising to observe that this results in a more balanced uptake of all nutrients, leading to a healthier plant, higher nutrient harvest in all types of soils and improving soil health. The present composition acts as a nutrient use efficient composition while meeting the need of crops by providing a multi nutritive solution with improved uptake by crops in a single application

On account of superior physical characteristics such as suspensibility, viscosity, pourability and spontaneity of dispersion exhibited, the composition of the present invention also finds a direct use in micro irrigation or drip irrigation systems.

### DESCRIPTION OF THE INVENTION

In describing the embodiment of the invention, specific terminology is chosen for the sake of clarity. However, it is not intended that the invention be limited to the specific terms so selected and it is to be understood that such specific terms include all technical equivalents that operate in a similar manner to accomplish a similar purpose. It is understood that any numerical range recited herein is intended to include all subranges subsumed. Also, unless denoted otherwise percentage of components in a composition are presented as weight percent. The term ‘parts’ and ‘percentage’ are interchangeable.

In some embodiments, the numerical parameters should be construed considering the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein.

The terms “a” or “an”, as used herein, are defined as one or more than one. The terms “including” and/or “having”, as used herein, are defined as comprising (i.e., open language).

The term “plant” or “crop” used in this application are interchangeable and wherever the term “plant” has been used shall also mean vegetations of similar nature namely crops, trees, shrub, herb etc.

According to the invention, the term liquid suspension encompasses suspension concentrate, oil dispersion or suspo-emulsion. The liquid as a vehicle can be water and/or a water miscible solvent or a water immiscible solvent or an oil.

As defined herein, the term “suspension concentrate” is a composition wherein solid particles are dispersed or suspended in a liquid. The term “suspension concentrates” or “aqueous suspension” or aqueous dispersion” or “an SC composition” can be used interchangeably. The liquid as a vehicle in the suspension concentrate can be water and/or a water miscible solvent. As defined herein, the term “oil dispersion” is a stable suspension of active ingredients in a water immiscible fluid/liquid or oil usually intended for dilution with water before use. The oil dispersion also further includes other dissolved ingredients in the formulation. The term “oil suspension” or “oil suspension concentrates” and “oil dispersion” can be used interchangeably.

As defined herein, the term suspo-emulsion is essentially a mixture of water-insoluble active constituents dispersed in a water-based solution; where one (or more) of the active constituents is a solid, formulated as a suspension form (SC) and one (or more) of the actives is an oil, formulated as an emulsion in water (EW).

The term ‘salts’ used in this application shall also encompass the compounds containing Zinc, Magnesium and Iron. The compounds of Zinc can include Zinc Oxide and the compounds of Magnesium can include Magnesium Oxide and the compounds of Iron can include Iron Oxide.

Oxides are compounds of metals such as Iron, Magnesium, and Zinc which are covered under salts of Iron, Magnesium and Zinc respectively.

D50 is the corresponding particle size when the cumulative percentage reaches 50%. D50 is also called as the median particle diameter or median particle size and represent average 50% of total particles to be smaller than the determined size.

D90 is used to indicate particle size distribution and represent average 90% of total particles to be smaller than the determined size. D90 is also the corresponding particle size when the cumulative percentage reaches 90%.

Nutrient Use Efficiency (NUE) is defined as a measure of how well plants use the available mineral nutrients. Improvement of NUE is an essential pre-requisite for expansion of crop production into marginal lands with low nutrient availability but also a way to reduce use of inorganic fertilizer.

A mixture is defined as a combination of two or more substances that are not chemically united to each other. A homogeneous mixture is defined as the one whose composition is uniform throughout the mixture. It is the type of mixture where the composition is constant throughout or the components that make up the mixture are distributed uniformly.

The present invention relates to a composition for crop nutrition in the form of liquid suspension comprising a homogeneous mixture of one or more of water insoluble Magnesium salt, complex or derivative thereof and one or more of water insoluble Zinc salt, complex or derivative thereof and one or more of water insoluble Iron salt, complex or derivative thereof with at least one agrochemically acceptable excipient. The liquid suspension composition is in the form of a suspension concentrate, an oil dispersion or a suspo-emulsion composition.

The liquid suspension composition of the present invention includes a homogeneous mixture of 1% to 50% by weight of one or more water insoluble Iron salt, complex or derivative thereof, 1% to 70% by weight of one or more water insoluble Magnesium salt, complexes or derivative thereof, 1% to 50% by weight of one or more water insoluble Zinc salt, complex or derivative thereof, and at least one agrochemically acceptable excipient, wherein elemental Iron is present in the range of 0.01% to 40% w/w of the total composition and wherein elemental Zinc is present in the range of 0.01% to 40% w/w of the total composition and wherein elemental Magnesium is present in the range of 0.01% to 50% w/w of the total composition. Further, the said crop nutrition composition comprises fine particles in the size range of 0.1 micron to 20 microns and exhibits improved suspensibility, viscosity, spontaneity of dispersion and pourability. According to an embodiment, the agrochemical excipient is structuring agent.

The present inventors surprisingly found that the present composition in the form of liquid suspension comprising Magnesium, Zinc and Iron together is not only effective but also synergistic. It was also noted by the present inventors that the application of the composition renders a greater and balanced uptake of not only Magnesium, Iron and Zinc but of other nutrients that remained entrapped in the soil and provide a natural bio-fortification solution in a sustainable manner, even in degraded soils.

It was observed that the surprising effect was noted when the present composition comprising a combination of water insoluble salts, complex or derivatives of Magnesium, Zinc and Iron in specific proportions was formulated into a liquid suspension along with a specific particle size distribution. The composition of the present invention was found to address the challenges of nutrient antagonism in the soil namely between Zinc and Iron, Magnesium and Zinc, Magnesium and Iron, etc. The present composition was further observed to prevent the leaching of these nutrients and make them available to the fullest extent for the uptake by crops and increase the overall yield.

According to an embodiment, the crop nutrition composition in the form of liquid suspension comprises particles in the size range of 0.1 micron to 20 microns, preferably the particles are in the size range of 0.1 micron to 15 microns. It was further observed that the present composition when formulated at a specific particle size of 0.1 micron to 20 microns, made the nutrients Magnesium, Zinc and Iron readily available for uptake by the plants and increase the overall yield. Thus, the particle size range of 0.1 micron to 20 microns of the crop nutrition composition was found to be important not only in terms of ease of application but also in terms of efficacy.

According to another embodiment, the crop nutrition composition of the present invention in the form of liquid suspension comprises particles having diameter distribution of D90 of about 15 microns, more preferably, the composition comprises particles having diameter distribution of D90 of about 10 microns.

According to a further embodiment, the water-insoluble Iron salts include one or more of but not limited to Iron Oxide, Iron Hydroxide, Iron Phosphate, Iron Fumarate, Iron Succinate, Iron Tartrate, Iron Sulphide, Iron Oxalate, Carbonyl Iron, Iron Silicate, Iron Rust, Limonite, Iron Carbonate, complexes, derivative and mixtures, thereof. The Iron Oxide includes but not limited to, Ferrous Oxide (FeO), Ferric Oxide (Fe₂O₃) or Red Oxide, and Ferroso Ferric Oxide (Fe₃O₄) or Black Iron Oxide. Iron Hydroxide includes, but is not limited to, Ferric Hydroxide, Yellow Iron Oxide (FeOOH), Iron Hydroxide (Fe(OH)₃), Iron Hydroxide (III), Iron Oxyhydroxide and Limonite. Iron Phosphate includes but not limited to, Ferric Phosphate, Ferric Phosphate Dihydrate, Ferric Phosphate Hydrate, Ferrous Pyrophosphate. Iron Fumarate includes but not limited to Ferrous Fumarate and Ferro Fumarate. Iron Succinate includes but is not limited to Ferrous Succinate and Succinic Acid Iron (II) salt. However, those skilled in the art will appreciate that it is possible to utilize other water-insoluble Iron salt, complex or derivative thereof without departing from the scope of the invention.

According to an embodiment, the water insoluble Iron salt, complex or derivative thereof include one or more of Iron containing minerals selected from but not limited to Iron ores including one or more of Roaldite, Taenite, Wüstite, Magnetite, Hematite, Troilite, Goethite, Greigite, Limonite, Siderite, Pyrite (Marcasite), Bernalite, Greenalite. However, the above list of ores or minerals is exemplary and not meant to limit the scope of the invention.

According to an embodiment, the water insoluble Iron salt, complex, derivative or mixture thereof is present in the range of 1% to 50% by weight of the total composition, wherein elemental Iron is present in the range of 0.01% to 40% by weight of the total composition. According to an embodiment, the water insoluble Iron salt, complex, derivative or mixture thereof is present in the range of 1% to 50% by weight of the total composition, wherein elemental Iron is present in the range of 0.01% to 38% by weight of the total composition. According to an embodiment, the water insoluble Iron salt, complex, derivative or mixture thereof is present in the range of 1% to 50% by weight of the total composition, wherein elemental Iron is present in the range of 0.01% to 34% by weight of the total composition.

According to an embodiment, the water insoluble Iron salt, complex, derivative or mixture thereof is present in the range of 1% to 50% by weight of the total composition. According to an embodiment, the water insoluble Iron salt, complex, derivative or mixture thereof is present in the range of 1% to 40% by weight of the total composition. According to an embodiment, the water insoluble Iron salt, complex, derivative or mixture thereof is present in the range of 1% to 30% by weight of the total composition. According to an embodiment, the water insoluble Iron salt, complex, derivative or mixture thereof is present in the range of 1% to 20% by weight of the total composition. According to an embodiment, the water insoluble Iron salt, complex, derivative or mixture thereof is present in the range of 1% to 10% by weight of the total composition. According to an embodiment, the water insoluble Iron salt, complex, derivative or mixture thereof is present in the range of 1% to 5% by weight of the total composition.

According to further embodiment, the water-insoluble Zinc salts include one or more of but not limited to Zinc Oxide, Zinc Carbonate, Zinc Sulphide, Zinc Molybdate, Zinc Phosphate, Zinc Nitrilotriacetic Acid (NTA), Zinc Borate, Zinc Silicate, Zinc Pyrophosphate, Zinc Citrate, complex or derivatives thereof. However, those skilled in the art will appreciate that it is possible to utilize other water insoluble Zinc salts, complexes or derivatives thereof without departing from the scope of the invention.

According to an embodiment, the water insoluble Zinc salt, complex or derivative thereof include one or more of Zinc containing minerals selected from but not limited to Zinc ores including one or more of Periclase, Danbaite, Ashoverite, Sphalerite, Wurtzite. However, the above list of ores or minerals is exemplary and not meant to limit the scope of the invention.

According to an embodiment, the water insoluble Zinc salt, complex, derivative or mixture thereof is present in the range of 1% to 50% by weight of the total composition, wherein elemental Zinc is present in the range of 0.01% to 40% by weight of the total composition. According to an embodiment, the water insoluble Zinc salt, complex, derivative or mixture thereof is present in the range of 1% to 50% by weight of the total composition, wherein elemental Zinc is present in the range of 0.01% to 34% by weight of the total composition. According to an embodiment, the water insoluble Zinc salt, complex, derivative or mixture thereof is present in the range of 1% to 50% by weight of the total composition, wherein elemental Zinc is present in the range of 0.01% to 31% by weight of the total composition.

According to an embodiment, the water insoluble Zinc salt, complex, derivative or mixture thereof is present in the range of 1% to 50% by weight of the total composition. According to an embodiment, the water insoluble Zinc salt, complex, derivative or mixture thereof is present in the range of 1% to 40% by weight of the total composition. According to an embodiment, the water-insoluble Zinc salt, complex, derivative or mixtures thereof is present in the range of 1% to 30% by weight of the total composition. According to an embodiment, the water-insoluble Zinc salt, complex, derivative or mixture thereof is present in the range of 1% to 20% by weight of the total composition. According to an embodiment, the water-insoluble Zinc salt, complex, derivative or mixture thereof is present in the range of 1% to 10% by weight of the total composition. According to an embodiment, the water insoluble Zinc salt, complex, derivative or mixture thereof is present in the range of 1% to 5% by weight of the total composition.

According to a further embodiment, the water insoluble Magnesium salts include one or more of but not limited to Magnesium Molybdate, Magnesium Hydroxide (Milk of Magnesia), Calcium Magnesium Phosphate, Magnesium Phosphate Tribasic, Magnesium Carbonate, Magnesium Aluminium Silicate, Calcium Magnesium Silicate, Magnesium Trisilicate, Magnesium Phosphate, Magnesium Silicate, Magnesium Oxide, complex, derivative thereof. However, those skilled in the art will appreciate that it is possible to utilize other water insoluble Magnesium salts, complexes or derivatives or mixtures thereof without departing from the scope of the invention.

According to an embodiment, the water insoluble Magnesium salts, complex or derivative thereof include one or more of Magnesium-containing minerals selected from but not limited to Magnesium ores including one or more of Periclase, Brucite, Sellaite, Kotoite, Pertsevite, Suanite, Magnesite, Szaibélyite, Neighborite. However, the above list of ores or minerals is exemplary and not meant to limit the scope of the invention.

According to an embodiment, the water insoluble Magnesium salt, complex, derivative or mixture thereof is present in the range of 1% to 70% by weight of the total composition, wherein elemental Magnesium is present in the range of 0.01% to 50% by weight of the total composition. According to an embodiment, the water insoluble Magnesium salt, complex, derivative or mixture thereof is present in the range of 1% to 70% by weight of the total composition, wherein elemental Magnesium is present in the range of 0.01% to 48% by weight of the total composition. According to an embodiment, the water insoluble Magnesium salt, complex, derivative or mixture thereof is present in the range of 1% to 70% by weight of the total composition, wherein elemental Magnesium is present in the range of 0.01% to 35% by weight of the total composition.

According to an embodiment, the water insoluble Magnesium salt, complex, derivative or mixture thereof is present in the range of 1% to 70% by weight of the total composition. According to an embodiment, the water insoluble Magnesium salt, complex, derivative or mixture thereof is present in the range of 1% to 60% by weight of the total composition. According to an embodiment, the water insoluble Magnesium salt, complex, derivative or mixture thereof is present in the range of 1% to 50% by weight of the total composition. According to an embodiment, the water insoluble Magnesium salt, complex, derivative or mixture thereof is present in the range of 1% to 40% by weight of the total composition. According to an embodiment, the water insoluble Magnesium salt, complex, derivative or mixture thereof is present in the range of 1% to 30% by weight of the total composition. According to an embodiment, the water insoluble Magnesium salt, complex, derivative or mixture thereof is present in the range of 1% to 20% by weight of the total composition. According to an embodiment, the water insoluble Magnesium salt, complex, derivative or mixture thereof is present in the range of 1% to 10% by weight of the total composition.

According to another embodiment, the crop nutrition composition in the form of liquid suspension comprises a homogeneous mixture of one or more of water insoluble Magnesium salt, complex or derivative thereof in the range of 1% to 70% by weight of the total composition and one or more of water insoluble Zinc salt, complex or derivative thereof in the range of 1% to 50% by weight of the total composition and one or more of water insoluble Iron salt, complex or derivative thereof in the range of 1% to 50% by weight of the total composition and one or more structuring agent in the range of 0.01% to 10% by weight of the total composition; wherein elemental Iron is present in the range of 0.01% to 40% w/w of the total composition and wherein elemental Zinc is present in the range of 0.01% to 40% w/w of the total composition and wherein elemental Magnesium is present in the range of 0.01% to 50% w/w of the total composition and wherein the composition comprises of particles in the size range of 0.1 micron to 20 microns.

According to an embodiment, the crop nutrition composition in the form of liquid suspension comprises a homogeneous mixture of one or more of Magnesium Oxide or Magnesium Silicate or Magnesium Carbonate or Magnesium Phosphate or Magnesium Hydroxide in the range of 1% to 70% by weight of the total composition and one or more of Zinc Oxide or Zinc Carbonate or Zinc Silicate or Zinc Hydroxide or Zinc Phosphate in the range of 1% to 50% by weight of the total composition and one or more of Iron Oxide or Iron Carbonate or Iron Hydroxide or Iron Silicate or Iron Phosphate in the range of 1% to 50% by weight of the total composition with one or more agrochemically acceptable excipient; wherein the composition comprises particles in the size range of 0.1 micron to 20 microns.

According to an embodiment, the crop nutrition composition in the form of liquid suspension comprises a homogeneous mixture of one or more of Magnesium Oxide or Magnesium Silicate or Magnesium Carbonate or Magnesium Phosphate or Magnesium Hydroxide in the range of 1% to 70% by weight of the total composition and one or more of Zinc Oxide or Zinc Carbonate or Zinc Silicate or Zinc Hydroxide or Zinc Phosphate in the range of 1% to 50% by weight of the total composition and one or more of Iron Oxide or Iron Carbonate or Iron Hydroxide or Iron Silicate or Iron Phosphate in the range of 1% to 50% by weight of the total composition with one or more structuring agent in the range of 0.01% to 10% by weight of the total composition, wherein the composition comprises particles in the size range of 0.1 micron to 20 microns.

According to an embodiment, the crop nutrition composition may further comprise at least one additional water insoluble plant nutrient.

According to an embodiment, the additional water insoluble plant nutrient is present in the range of from 0.01% to 40% by weight of the total composition.

According to an embodiment, the crop nutrition composition is devoid of fertilizers that primarily comprise of alginic acid or urea.

According to an embodiment, the crop nutrition composition in the form of liquid suspension comprises at least one agrochemical excipient. According to further embodiment, the agrochemically acceptable excipients which are used in liquid suspension composition include at least one surfactant, dispersing agent, wetting agent, humectants, solvents, spreading agent, suspending agents or suspension aid, penetrating agent, sticking agents, drift reducing agents, ultraviolet absorbents, UV ray scattering agents, preservatives, stabilizer, buffers or pH adjusters or neutralizing agents, antifreezing agent or freezing point depressants, antifoaming agents, structuring agents, anticaking agent. However, those skilled in the art will appreciate that it is possible to utilize additional agrochemically acceptable excipients without departing from the scope of the present invention.

According to an embodiment, the agrochemical excipients are present in a concentration range of 0.01% to 97% by weight of the total composition. According to an embodiment, the agrochemical excipients are present in a concentration range of at least 96% by weight of the total composition. According to an embodiment, the agrochemical excipients are present in a concentration range of at least 95% by weight of the total composition. According to an embodiment, the agrochemical excipients are present in a concentration range of at least 90% by weight of the total composition. According to an embodiment, the agrochemical excipients are present in a concentration range of at least 75% by weight of the total composition. According to an embodiment, the agrochemical excipients are present in a concentration range of at least 55% by weight of the total composition. According to an embodiment, the agrochemical excipients are present in a concentration range of at least 35% by weight of the total composition. According to an embodiment, the agrochemical excipients are present in a concentration range of at least 25% by weight of the total composition. According to an embodiment, the agrochemical excipients are present in a concentration range of at least 15% by weight of the total composition. According to an embodiment, the agrochemical excipients are present in a concentration range of at least 5% by weight of the total composition. According to an embodiment, the agrochemical excipients are present in a concentration range of at least 1% by weight of the total composition. According to an embodiment, the agrochemical excipients are present in a concentration range of at least 0.1% by weight of the total composition

According to an embodiment, the surfactants which are used in the crop nutrition composition include one or more of emulsifiers, wetting agents and dispersing agents. According to an embodiment, the surfactants which are used in the composition include one or more of anionic, cationic, non-ionic, amphoteric and polymeric surfactants.

The anionic surfactants include one or more of, but not limited to a salt of Fatty Acid, a Benzoate, a Polycarboxylate, a salt of Alkylsulfuric Acid Ester, Alkyl Ether Sulfates, an Alkyl Sulfate, an Alkylarylsulfate, an Alkyl Diglycol Ether Sulfate, a Salt of Alcohol Sulfuric Acid Ester, an Alkyl Sulfonate, an Alkylaryl Sulfonate, an Aryl Sulfonate, a Lignin Sulfonate, an Alkyl Diphenyl Ether Disulfonate, a Polystyrene Sulfonate, a Salt Of Alkylphosphoric Acid Ester, an Alkylaryl Phosphate, a Styrylaryl Phosphate, Sulfonate Docusates, a Salt Of Polyoxyethylene Alkyl Ether Sulfuric Acid Ester, a Polyoxyethylenealkylaryl Ether Sulfate, Alkyl Sarcosinates, Alpha Olefin Sulfonate Sodium Salt, Alkyl Benzene Sulfonate or Its Salts, Sodium Lauroylsarcosinate, Sulfosuccinates, Polyacrylates, Polyacrylates-Free Acid and Sodium Salt, Salt of Polyoxyethylenealkylaryl Ether Sulfuric Acid Ester, a Polyoxyethylene Alkyl Ether Phosphate, a Salt of Polyoxyethylenealkylaryl Phosphoric Acid Ester, Sulfosuccinates-M ono and other Diesters, Phosphate Esters, Alkyl Naphthalene Sulfonate-Isopropyl and Butyl Derivatives, Alkyl Ether Sulfates-Sodium And Ammonium Salts; Alkyl Aryl Ether Phosphates, Ethylene Oxides and Its Derivatives, a salt of Polyoxyethylene Aryl Ether Phosphoric Acid Ester, Mono-Alkyl Sulphosuccinates, Aromatic Hydrocarbon Sulphonates, 2-Acrylamido-2-Methylpropane Sulfonic Acid, Ammonium Laurylsulphate, Docusate, Disodium Cocoamphodiacetate, Magnesium Laurethsulfate, Phospholipid, Potassium Lauryl Sulfate, Soap, Soap Substitute, Sodium Alkyl Sulfate, Sodium Dodecyl Sulfate, Sodium Dodecylbenzenesulfonate, Sodium Laurate, Sodium Laurethsulfate, Sodium Lauroylsarcosinate, Sodium Myrethsulfate, Sodium Nonanoyloxybenzenesulfonate, Alkyl Carboxylates, Sodium Stearate, Alpha Olefin Sulphonates, Naphthalene Sulfonate Salts, Alkyl Naphthalene Sulfonate Fatty Acid salts, Naphthalene Sulfonate Condensates-Sodium salt, Fluoro Carboxylate, Fatty Alcohol Sulphates, Alkyl Naphthalene Sulfonate Condensates-Sodium Salt, A Naphthalene Sulfonic Acid Condensed with Formaldehyde or a Salt of Alkylnaphthalene Sulfonic Acid condensed with Formaldehyde or salts or derivatives thereof.

The non-ionic surfactants include one or more of but not limited to Polyol Esters, Polyol Fatty Acid Esters, Polyethoxylated Esters, Polyethoxylated Alcohols, Ethoxylated and Propoxylated Fatty Alcohols, Ethoxylated and Propoxylated Alcohols, Ethylene Oxide (EO)/Propylene Oxide (PO) Copolymers; EO and PO Block Copolymers, Di, Tri-Block Copolymers; Block Copolymers Of Polyethylene Glycol and Polypropylene Glycol, Poloxamers, Polysorbates, Alkyl Polysaccharides such as Alkyl Polyglycosides and Blends thereof, Amine Ethoxylates, Sorbitan Fatty Acid Ester, Glycol and Glycerol Esters, Glucosidyl Alkyl Ethers, Sodium Tallowate, Polyoxyethylene Glycol, Sorbitan Alkyl Esters, Sorbitan Derivatives, Fatty Acid Esters of Sorbitan (Spans) and Their Ethoxylated Derivatives (Tweens), and Sucrose Esters of Fatty Acids, Cocamide Diethanolamine (DEA), Cocamide Monoethanolamine (MEA), Decyl Glucoside, Decylpolyglucose, Glycerol Monostearate, Lauryl Glucoside, Maltosides, Monolaurin, Narrow-Range Ethoxylate, Nonidet P-40, Nonoxynol-9, Nonoxynols, Octaethylene Glycol Monododecyl Ether, N-Octyl Beta-D-Thioglucopyranoside, Octyl Glucoside, Oleyl Alcohol, PEG-10 Sunflower Glycerides, Pentaethylene Glycol Monododecyl Ether, Polidocanol, Poloxamer, Poloxamer 407, Polyethoxylated Tallow Amine, Polyglycerol Polyricinoleate, Polysorbate, Polysorbate 20, Polysorbate 80, Sorbitan, Sorbitanmonolaurate, Sorbitanmonostearate, Sorbitantristearate, Stearyl Alcohol, Surfactin, Glyceryl Laureate, Lauryl Glucoside, Nonylphenolpolyethoxyethanols, Nonyl Phenol Polyglycol Ether, Castor Oil Ethoxylate, Polyglycol Ethers, Polyadducts of Ethylene Oxide and Propylene Oxide, Block Copolymer of Polyalkylene Glycol Ether and Hydroxystearic Acid, Tributylphenoxypolyethoxy Ethanol, Octylphenoxypolyethoxy Ethanol, Etho-Propoxylatedtristyrlphenols, Ethoxylated Alcohols, Polyoxy Ethylene Sorbitan, Fatty Acid Polyglyceride, a Fatty Acid Alcohol Polyglycol Ether, Acetylene Glycol, Acetylene Alcohol, an Oxyalkylene Block Polymer, Polyoxyethylene Alkyl Ether, Polyoxyethylenealkylaryl Ether, a Polyoxyethylenestyrylaryl Ether, a Polyoxyethylene Glycol Alkyl Ether, Polyethylene Glycol, a Polyoxyethylene Fatty Acid Ester, a Polyoxyethylenesorbitan Fatty Acid Ester, a Polyoxyethyleneglycerin Fatty Acid Ester, Alcohol Ethoxylates-C6 to C16/18 Alcohols, Linear and Branched, Alcohol Alkoxylates-Various Hydrophobes and EO/PO Contents and Ratios, Fatty Acid Esters-M ono and Diesters, Lauric, Stearic and Oleic, Glycerol Esters—with and without EO, Lauric, Stearic, Cocoa and Tall Oil Derived, Ethoxylated Glycerine, Sorbitan Esters—with and without EO; Lauric, Stearic and Oleic Based, Mono and Trimesters, Castor Oil Ethoxylates-5 to 200 Moles EO, Non-Hydrogenated and Hydrogenated, Block Polymers, A mine Oxides-Ethoxylated and Non-Ethoxylated; Alkyl Dimethyl, Fatty Amine Ethoxylates-Coco, Tallow, Stearyl, Oleyl Amines, a Polyoxyethylene Hydrogenated Castor Oil or a Polyoxypropylene Fatty Acid Ester, salts or derivatives thereof.

Amphoteric or Zwitterionic surfactants include one or more of, but not limited to one or more of Betaine, Coco and Lauryl Amidopropyl Betaines, Coco Alkyl Dimethyl Amine Oxides, Alkyl Dimethyl Betaines; C8 to C18, Alkyl Dipropionates-Sodium Lauriminodipropionate, Cocoamidopropyl Hydroxyl Sulfobetaine, Imidazolines, Phospholipids Phosphatidylserine, Phosphatidylethanolamine, Phosphatidylcholine and Sphingomyelins, Lauryl Dimethylamine Oxide, Alkyl Amphoacetates and Proprionates, Alkyl Ampho(Di)Acetates and Di-Proprionates, Lecithin and Ethanolamine Fatty Amides or salts, derivatives thereof.

Surfactants that are commercially available under the trademark but are not limited to one or more of Atlas G5000, TERMUL 5429, TERMUL 2510, ECOTERIC®, EULSOGEN® 118, Genapol®X, Genapol®OX-080, Genapol® C 100, Emulsogen® EL 200, Arlacel P135, Hypermer 8261, Hypermer B 239, Hypermer B261, Hypermer B246sf, Solutol HS 15, Promulgen™ D, Soprophor 7961P, Soprophor TSP/461, Soprophor TSP/724, Croduret 40, Etocas 200, Etocas 29, Rokacet R26, Cetomacrogol 1000, CHEMONIC OE-20, Triton N-101, Triton X-100, Tween 20, 40, 60, 65, 80, Span20, 40, 60, 80, 83, 85, 120, Brij®, Atlox 4912, Atlas G5000, TERMUL 3512, TERMUL 3015, ECOTERIC® T85, ECOTERIC® T20, TERIC 12A4,] IGEPAL CA-630 and Isoceteth-20.

However, those skilled in the art will appreciate that it is possible to utilize other conventionally known surfactants without departing from the scope of the present invention. The surfactants are commercially manufactured and available through various companies.

According to an embodiment, the surfactant is present in an amount of 0.1% to 40% w/w of the total composition. According to an embodiment, the surfactant is present in an amount of 0.1% to 30% w/w of the total composition. According to further embodiment, the surfactant is present in an amount of 0.1% to 20% w/w of the total composition. According to further embodiment, the surfactant is present in an amount of 0.1% to 10% w/w of the total composition.

According to an embodiment, the dispersing agents which are used in the crop nutrition composition includes, but not limited to one or more of polyvinyl pyrrolidone, polyvinyl alcohol, lignin sulphonates, phenol naphthalene sulphonates, alkali metal, alkaline earth metal and ammonium salts of lignosulfonic acid, lignin derivatives, alkyl sulfates, alkylsulfonates, fatty alcohol sulfates, fatty acids and sulfated fatty alcohol glycol ethers, polyoxyethylene alkyl ethers, lauryl sulfate, polyoxyethylene alkyl ether sulphate, polyoxyethylenestyryl phenyl ether sulfate ester salts and the like, alkali metal salts thereof, ammonium salts or amine salts, polyoxyethylene alkyl phenyl ether, polyoxyethylenestyryl phenyl ether, polyoxyethylene alkyl esters, or polyoxyethylenesorbitan alkyl esters, and the like, mixture of sodium salt of naphthalene sulphonic acid urea formaldehyde condensate and sodium salt of phenol sulphonic formaldehyde condensate ethoxylated alkyl phenols, ethoxylated fatty acids, alkoxylated linear alcohols, polyaromatic sulfonates, sodium alkyl aryl sulfonates, glyceryl esters, ammonium salts of maleic anhydride copolymers, maleic anhydride copolymers, phosphate esters, condensation products of aryl sulphonic acids and formaldehyde, addition products of ethylene oxide and fatty acid esters, salts of addition products of ethylene oxide and fatty acid esters, sodium salt of isodecylsulfosuccinic acid half ester, polycarboxylates, sodium alkyl benzene sulfonates, sodium salts of sulfonated naphthalene, ammonium salts of sulfonated naphthalene, salts of polyacrylic acids, sodium salts of condensed phenolsulfonic acid as well as the naphthalene sulfonate-formaldehyde condensates, sodium naphthalene sulfonate formaldehyde condensates, tristyrylphenolethoxylate phosphate esters, aliphatic alcohol ethoxylates, alkyl fatty acids, alkoxylated linear alcohols, polyaromatic sulfonates, sodium alkyl aryl sulfonates, glyceryl esters, ammonium salts of maleic anhydride copolymers, maleic anhydride copolymers, phosphate esters, condensation products of aryl sulphonic acids and formaldehyde, addition products of ethylene oxide and fatty acid esters, salts of addition products of ethylene oxide and fatty acid esters, sodium salt of isodecylsulfosuccinic acid half ester, polycarboxylates, sodium alkyl benzene sulfonates, sodium salts of sulfonated naphthalene, ammonium salts of sulfonated naphthalene, salts of polyacrylic acids, sodium salts of condensed phenolsulfonic acid as well as the naphthalene sulfonate formaldehyde condensates, sodium naphthalene sulfonate formaldehyde condensates, tristyrylphenolethoxylate phosphate esters, aliphatic alcohol ethoxylates, alkyl ethoxylates, EO-PO block copolymers, graft copolymers, ammonium salts of sulfonated naphthalene, salts of polyacrylic acids or salts or derivatives thereof.

Commercially available dispersing agents include “Morwet D425” (sodium naphthalene formaldehyde condensate ex Nouryon, USA), Sulfated Alkyl Carboxylate and Alkyl Naphthalene Sulfonate—Sodium Salt, “Tamol PP” (sodium salt of a phenolsulphonic acid condensate), “Reax 80N” (sodium lignosulphonate), “Wettol D1” sodium alkylnaphthalene sulphonate (ex BASF). However, those skilled in the art will appreciate that it is possible to utilize other conventionally known dispersants without departing from the scope of the present invention. The dispersing agents are commercially manufactured and available through various companies.

According to an embodiment, the dispersing agent is present in an amount of 0.1%-40% w/w of the total composition. According to an embodiment, the dispersing agent is present in an amount of 0.1%-30% w/w of the total composition. According to an embodiment, the dispersing agent is present in an amount of 0.1%-20% w/w of the total composition.

According to an embodiment the wetting agents used in the crop nutrition composition include, but not limited to one or more of phenol naphthalene sulphonates, alkyl naphthalene sulfonate, sodium alkyl naphthalene sulfonate, naphthalene sulphonate sodium salt, sodium salt of sulfonated alkylcarboxylate, polyoxyalkylated ethyl phenols, dibutylnaphthalene-sulfonic acid, alkylarylsulfonates, dioctyl sulfosuccinate, polyoxyethoxylated fatty alcohols, polyoxyethoxylated fatty amines, lignin derivatives, alkane sulfonates, alkylbenzene sulfonates, salts of polycarboxylic acids, salts of esters of sulfosuccinic acid, alkylpolyglycol ether sulfonates, alkyl ether phosphates, alkyl ether sulphates and alkyl sulfosuccinic monoesters, salts, derivatives thereof. However, those skilled in the art will appreciate that it is possible to utilize other conventionally known wetting agents without departing from the scope of the present invention. The wetting agents are commercially manufactured and available through various companies.

According to an embodiment, the wetting agent is present in an amount of 0.1%-30% w/w of the total composition. According to an embodiment, the wetting agent is present in an amount of 0.1%-20% w/w of the total composition. According to an embodiment, the wetting agent is present in an amount of 0.1%-10% w/w of the total composition.

According to an embodiment, the solvents used in the crop nutrition composition include water miscible solvents, water immiscible solvents or oils.

The water miscible solvents include, but are not limited to one or more of 1, 4-Dioxane, Ethylene glycol, Glycerol, N-Methyl-2-pyrrolidone, 1,3-Propanediol, 1,5-Pentanediol, Propylene glycol, Triethylene glycol, 1,2-Butanediol, 1,3-Butanediol, 1,4-Butanediol, Dimethylformamide, Dimethoxyethane, Dimethyloctanamide, Dimethyldecanamide. However, those skilled in the art will appreciate that it is possible to utilize other water miscible solvents without departing from the scope of the present invention.

According to an embodiment, water immiscible solvents include one or more of aromatic and non-aromatic hydrocarbons, halogenated aromatic and non-aromatic hydrocarbons, petroleum distillates, aromatic and non-aromatic ethers, esters or amides, oils or mixtures thereof. According to further embodiment, the oils can be one or more of a mineral oil, petroleum oil, vegetable oil or animal oil or derivatives or mixtures thereof. However, those skilled in the art will appreciate that it is possible to utilize other water immiscible solvents without departing from the scope of the present invention.

The mineral oil or petroleum oil is one or more of aliphatic or isoparaffinic series, and mixtures of aromatic and aliphatic hydrocarbons; halogenated aromatic or aliphatic hydrocarbons. Paraffinic oil can be selected from linear or branched C8 to C30 paraffins for example such as octane, nonane, decane, undecane, dodecane, tridecane, tetradecane, pentadecane, hexadecane, their mixtures, or mixtures thereof with higher boiling homologs, such as hepta-, octa-, nona-decane, eicosane, heneicosane, docosane, tricosane, tetracosane, pentacosane, and the branched chain isomers thereof, unsubstituted or substituted aromatic or cycloaliphatic C7 to C18 hydrocarbon compounds such as mono- or polyalkyl-substituted benzenes, or mono- or polyalkyl-substituted naphthalenes, or transesterification products thereof, liquid esters of C1 to C12 alcohols such as butanol, n-octanol, i-octanol, dodecanoi, cyclopentanol, cyclohexanol, cyclooctanol, ethylene glycol or propylene glycol with C2 to C12 carboxylic or polycarboxylic acids, such as caproic acid, capric acid, caprylic acid, pelargonic acid, succinic acid and glutaric acid; or with aromatic carboxylic acids such as benzoic acid, toluic acid, salicylic acid and phthalic acid, liquid amides of C1 to C5 amines, alkylamines or alkanolamines with C6 to C18 carboxylic acids, or derivatives thereof. Esters which can be used in the oil dispersions of the invention are benzyl acetate, caproic acid ethyl ester, pelargonic acid ethyl ester, benzoic acid methyl or ethyl ester, salicylic acid methyl, propyl, or butyl ester, diesters of phthalic acid with saturated aliphatic or alicyclic C1 to C12 alcohols, such as phthalic acid dimethyl ester, dibutyl ester, diisooctyl ester, or liquid amides of C1-C3 amines, alkylamines or alkanolamines with C6-C18 carboxylic acids or derivatives or mixtures thereof. However, those skilled in the art will appreciate that it is possible to utilize other mineral or petroleum oils without departing from the scope of the present invention.

The vegetable oil include one or more of seed oil. The vegetable oil also includes one or more of soy bean oil, rape seed oil, olive oil, castor oil, sunflower seed oil, coconut oil, corn oil, cotton seed oil, linseed oil, palm oil, peanut oil, safflower oil, sesame oil, kapok oil, papaya oil, camellia oil, rice bran oil, tung oil and the like; and esters of the above vegetable oils, or transesterification products thereof such as soy bean oil methyl esters, ethyl esters, propyl esters, butyl esters or derivatives thereof. The animal oil include one or more of whale oil, cod-liver oil, or mink oil. However, those skilled in the art will appreciate that it is possible to utilize other vegetable or animal oils without departing from the scope of the present invention.

The petroleum distillates include one or more of aromatic hydrocarbons derived from benzene, such as toluene, xylenes, other alkylated benzenes and the like, and naphthalene derivatives, aliphatic hydrocarbons such as hexane, octane, cyclohexane, and the like, mineral oils from the aliphatic or isoparaffinic series, and mixtures of aromatic and aliphatic hydrocarbons; halogenated aromatic or aliphatic hydrocarbons; vegetable, seed or animal oils such as soybean oil, rape seed oil, olive oil, castor oil, sunflower seed oil, coconut oil, corn oil, cotton seed oil, linseed oil, palm oil, peanut oil, safflower oil, sesame oil, tung oil and the like, and C₁-C₆ mono-esters derived from vegetable oils such as methyl oleate, methyl soyate, and methyl laurate, seed or animal oils; C₁-C₆ dialkyl amides of C₆-C₂₀ saturated and unsaturated aliphatic carboxylic acids; C₁-C₁₂ esters of aromatic carboxylic acids and dicarboxylic acids and C₁-C₁₂ esters of aliphatic and cyclo-aliphatic carboxylic acids; C₄-C₁₂ polyesters of dihydric, trihydric, or other lower polyalcohols such as, propylene glycol dioleate, di-octyl succinate, di-butyl adipate, di-octyl phthalate and the like.

According to an embodiment, the composition includes organic solvents or co-solvents such as ethers like tetrahydrofuran and the like, alkylene glycol dialkyl ethers such as ethylene glycol diethyl ether and the like, amides such as dimethylformamide, dimethylacetamide or N-methylpyrrolidone and the like, ketones such as methyl ethyl ketone and the like, nitriles such as butyronitrile and the like, sulfoxides or sulfones such as dimethyl sulfoxide or sulfolane and the like, and alkylene carbonates such as propylene or butylene carbonate. However, those skilled in the art will appreciate that it is possible to utilize other organic solvents or co-solvents without departing from the scope of the present invention.

According to an embodiment, the solvent is present in an amount of 0.1-95% w/w of the total composition. According to an embodiment, the solvent is present in an amount of 0.1-60% w/w of the total composition. According to an embodiment, the solvent is present in an amount of 0.1-40% w/w of the total composition. According to an embodiment, the solvent is present in an amount of 0.1-30% w/w of the total composition.

According to an embodiment, the carriers which are used in the crop nutrition composition include, but are not limited to one or more of solid carriers or fillers or diluents. According to another embodiment, the carriers include mineral carriers, plant carriers, synthetic carriers, water-soluble carriers. However, those skilled in the art will appreciate that it is possible to utilize different carriers without departing from the scope of the present invention. The carriers are commercially manufactured and available through various companies.

The solid carriers include natural minerals like clay such as china clay, acid clay, kaolin such as kaolinite, dickite, nacrite, and halloysite, serpentines such as chrysotile, lizardite, antigorite, amesite, synthetic and diatomaceous silicas, montmorillonite minerals such as sodium montmorillonite, smectites, such as saponite, hectorite, sauconite, hyderite, micas, such as pyrophyllite, talc, agalmatolite, muscovite, phengite, sericite, and illite, silicas such as cristobalite and quartz, such as attapulgite and sepiolite, vermiculite, laponite, pumice, bauxite, hydrated aluminas, perlite, sodium bicarbonate, volclay, limestone, natural and synthetic silicates, charcoal, silicas, wet process silicas, dry process silicas, calcined products of wet process silicas, surface-modified silicas, mica, zeolite, diatomaceous earth, derivatives thereof, chalks (Omya®), fuller's earth, loess, mirabilite, white carbon, slaked lime, synthetic silicic acid, starch, modified starch (Pineflow, available from Matsutani Chemical industry Co., Ltd.), cellulose, plant carriers such as cellulose, chaff, wheat flour, wood flour, starch, rice bran, wheat bran, and soyabean flour, casein sodium, sucrose, salt cake, potassium pyrophosphate, sodium tripolyphosphate or derivatives or mixtures thereof. Commercially available Silicates are A erosil brands, Sipernat brands as Sipernat® 50S and CALFLO E and kaolin 1777. However, those skilled in the art will appreciate that it is possible to utilize different solid carriers without departing from the scope of the present invention. The solid carriers are commercially manufactured and available through various companies.

According to an embodiment, the carrier is present in an amount of 0.1% to 97% w/w of the composition. According to further embodiment, the carrier is present in an amount of 0.1% to 80% w/w of the composition. According to further embodiment, the carrier is present in an amount of 0.1% to 60% w/w of the composition. According to further embodiment, the carrier is present in an amount of 0.1% to 40% w/w of the composition. According to further embodiment, the carrier is present in an amount of 0.1% to 20% w/w of the composition.

According to an embodiment, the antifoaming agents or defoamers which are used in the crop nutrition composition include, but not limited to one or more of silica, siloxane, silicone dioxide, polydimethyl siloxane, alkyl polyacrylates, ethylene oxide/propylene oxide copolymers, polyethylene glycol, silicone oils and magnesium stearate or derivatives thereof. Preferred antifoaming agents include silicone emulsions (such as, e.g., Silikon® SRE, Wacker or Rhodorsil® from Rhodia), long-chain alcohols, fatty acids, fluoro-organic compounds. However, those skilled in the art will appreciate that it is possible to utilize other conventionally known antifoaming agents without departing from the scope of the present invention. The antifoaming agents are commercially manufactured and available through various companies.

According to an embodiment, the anti-foaming agent is present in an amount of 0.01% to 20% w/w of the total composition.

According to an embodiment, the pH-adjusters or buffers or neutralizing agents which are used in the composition include both acids and bases of the organic or inorganic type and mixtures thereof. According to further embodiment, pH-adjusters or buffers or neutralizing agents include, but not limited to one or more of organic acids, inorganic acids and alkali metal compounds or salts, derivatives thereof. According to an embodiment, the organic acids include, but not limited to one or more of citric, malic, adipic, fumaric, maleic, succinic, and tartaric acid, or salts, derivatives thereof, and the mono-, di-, or tribasic salts of these acids or derivatives thereof. Alkali metal compounds include, but not limited to one or more of hydroxides of alkali metals such as sodium hydroxide and potassium hydroxide, carbonates of alkali metals such as sodium carbonate, hydrogen carbonates of alkali metals such as sodium hydrogen carbonate and alkali metal phosphates such as sodium phosphate and mixtures thereof. According to an embodiment, the salts of inorganic acids include, but not limited to one or more of alkali metal salts such as, sodium chloride, potassium chloride, sodium nitrate, potassium nitrate, sodium sulfate, potassium sulfate, sodium monohydrogen phosphate, potassium monohydrogen phosphate, sodium dihydrogen phosphate, potassium dihydrogen phosphate and the like. Mixtures can also be used to create a pH-adjusters or buffers or neutralizing agents. However, those skilled in the art will appreciate that it is possible to utilize other conventionally known pH-adjusters or buffers or neutralizing agents without departing from the scope of the present invention. The pH-adjusters or buffers or neutralizing agents are commercially manufactured and available through various companies.

According to an embodiment, the pH-adjusters or buffers are present in an amount of 0.01% to 20% w/w of the total composition. According to an embodiment, the pH-adjusters or buffers are present in an amount of 0.01% to 10% w/w of the total composition. According to an embodiment, the pH-adjusters or buffers are present in an amount of 0.01% to 5% w/w of the total composition. According to an embodiment, the pH-adjusters or buffers are present in an amount of 0.01% to 1% w/w of the total composition.

According to an embodiment, the anticaking agents which are used in the crop nutrition composition include, but are not limited to one or more of polysaccharides such as as starch, alginic acid, mannose, galactose; poly(vinylpyrrolidone), fumed silica (white carbon), ester gum, a petroleum resin, Foammaster® Soap L sodium stearate, Brij® 700 polyoxyethylene (100) stearylether, sodium acetate, sodium metasilicate, sodium alkylsulfosuccinates, sodium carbonate or bicarbonate, salts or derivatives thereof. However, those skilled in the art will appreciate that it is possible to utilize different anti caking agents without departing from the scope of the present invention. The anti-caking agents are commercially manufactured and available through various companies.

According to an embodiment, the anticaking agents is present in an amount of 0.1% to 20% w/w of the total composition. According to an embodiment, the anticaking agents is present in an amount of 0.1% to 15% w/w of the total composition. According to an embodiment, the anticaking agents is present in an amount of 0.1% to 10% w/w of the total composition.

According to an embodiment, the spreading agents which are used in the composition include, but not limited to one or more of a copolymer of maleic acid with a styrene compound, a (meth)acrylic acid copolymer, a half ester of a polymer consisting of polyhydric alcohol with dicarboxylic anhydride, a water-soluble salt of polystyrene sulfonic acid, fatty acids, latex, aliphatic alcohols, vegetable oils such as cottonseed or inorganic oils, petroleum distillates, modified trisiloxanes, polyglycol or salts or derivatives thereof. However, those skilled in the art will appreciate that it is possible to utilize other conventionally known spreading agents without departing from the scope of the present invention. The spreading agents are commercially manufactured and available through various companies.

According to an embodiment, the spreading agent is present in an amount of 0.01% to 20% w/w of the total composition. According to an embodiment, the spreading agent is present in an amount of 0.01% to 5% w/w of the total composition.

According to an embodiment, the sticking agents which are used in the composition include, but not limited to one or more of paraffin, a polyamide resin, polyacrylate, polyoxyethylene, wax, polyvinyl alkyl ether, an alkylphenol-formalin condensate, fatty acids, latex, polyvinyl pyrrolidone, aliphatic alcohols, gums such as xanthan gum, gum ghati, gum arabic etc, vegetable oils such as cottonseed, or inorganic oils, petroleum distillates, modified trisiloxanes, polyglycol, a synthetic resin emulsion or salts or derivatives thereof. However, those skilled in the art will appreciate that it is possible to utilize other conventionally known sticking agents without departing from the scope of the present invention. The sticking agents are commercially manufactured and available through various companies.

According to an embodiment, the sticking agent is present in an amount of 0.01% to 30% w/w of the total composition. According to an embodiment, the sticking agent is present in an amount of 0.01% to 15% w/w of the total composition.

According to an embodiment, the structuring agents which are used in the crop nutrition composition include, but not limited to one or more of thickeners, viscosity modifiers, tackifiers, suspension aids, rheological modifiers or anti-settling agents. A structuring agent prevents sedimentation of the active ingredient particles after prolonged storage.

According to an embodiment, the structuring agents which are used in the composition include, but not limited to one or more polymers such as polyacrylics, polyacrylamides, polysaccharides, hydrophobically modified cellulose derivatives, co-polymers of cellulose derivatives, carboxyvinyl or polyvinyl pyrrolidones, polyethylenes, polyethylene oxide, polyvinyl alcohol and derivatives; clays such as bentonite clays, kaolin, smectite, attapulgites, attaclays with high surface area silica and natural gums such as guar gum, xanthan gum, gum Arabic, gum tragacanth, rhamsan gum, locust bean gum, carageenan, welan gum, veegum, gelatin, dextrin, collagen; polyacrylic acids and their sodium salts; the polyglycol ethers of fatty alcohols and polyethylene oxide or polypropylene oxide condensation products and mixtures thereof and include ethoxylated alkyl phenols (also designated in the art as alkylaryl polyether alcohols); ethoxylated aliphatic alcohols (or alkyl polyether alcohols); ethoxylated fatty acids (or polyoxyethylene fatty acid esters); ethoxylatedanhydrosorbitol esters (or polyethylene sorbitan fatty acid esters), long chain amine and cyclic amine oxides which are nonionic in basic solutions; long chain tertiary phosphine oxides; and long chain dialkyl sulfoxides, fumed silica, mixture of fumed silica and fumed aluminium oxide, swellable polymers, polyamides or its derivatives; polyols such as glycerine, poly(vinyl acetate), sodium polyacrylate, poly(ethylene glycol), phospholipid (for example, cephalin, and the like); stachyose, fructo-oligosaccharides, amylose, pectins, alginates, hydrocolloids and mixtures thereof. Also, celluloses such as hemicellulose, carboxymethylcellulose, ethylcellulose, hydroxyethylcellulose, hydroxy-methyl ethyl cellulose, hydroxyl ethyl propyl cellulose, methylhydroxyethylcellulose, methylcellulose; starches such, starch acetates, starch hydroxyethyl ethers, ionic starches, long-chain alkyl starches, dextrins, maltodextrin, corn starch, amine starches, phosphates starches, and dialdehyde starches; plant starches such as corn starch and potato starch; other carbohydrates such as pectin, dextrin, amylopectin, xylan, glycogen, agar, gluten, alginic acid, phycocolloids, chitin, or derivatives thereof. However, those skilled in the art will appreciate that it is possible to utilize other conventionally known structuring agents without departing from the scope of the present invention.

Preferred structuring agents include one or more of xanthan gum, aluminium silicate, Hydroxypropyl methyl cellulose, carboxymethyl cellulose, methylcellulose, polysaccharide, alkaline earth metal silicate, clays such as bentonite clay, gelatin, and polyvinyl alcohol. The structuring agents are commercially manufactured and available through various companies.

According to an embodiment, the structuring agent is present in an amount of 0.01% to 10% w/w of the composition. According to an embodiment, the structuring agent is present in an amount of 0.01% to 5% w/w of the composition. According to an embodiment, the structuring agent is present in an amount of 0.01% to 4% w/w of the composition. According to an embodiment, the structuring agent is present in an amount of 0.01% to 3% w/w of the composition. According to an embodiment, the structuring agent is present in an amount of 0.01% to 2% w/w of the composition. According to an embodiment, the structuring agent is present in an amount of 0.01% to 1% w/w of the composition. According to an embodiment, the structuring agent is present in an amount of 0.01% to 0.1% w/w of the composition.

According to an embodiment, the anti-freezing agents or freezing point depressants used in the composition include, but are not limited to one or more of polyhydric alcohols such as ethylene glycol, diethylene glycol, dipropylene glycol, propylene glycol, butyrolactone, N,N-dimethyl-formamide, glycerol, monohydric or polyhydric alcohols, glycol ethers, glycol ethers, glycol monoethers such as the methyl, ethyl, propyl and butyl ether of ethylene glycol, diethylene glycol, propylene glycol and dipropylene glycol, glycol diethers such as methyl and ethyl diethers of ethylene glycol, diethylene glycol and dipropyleneglycol or urea, glycerol, isopropanol, propylene glycol monomethyl ether, di- or tripropylene glycol monomethyl ether or cyclohexanol, carbohydrates such as glucose, mannose, fructose, galactose, sucrose, lactose, maltose, xylose, arabinose, sorbitol, mannitol, trehalose, raffinose or derivatives thereof. However, those skilled in the art will appreciate that it is possible to utilize different anti-freezing agents without departing from the scope of the present invention. The anti-freezing agents are commercially manufactured and available through various companies.

According to an embodiment, the chelating or complexing or sequestering agents which are used in the composition include, but not limited to one or more of polycarboxylic acids such as polyacrylic acid and the various hydrolyzed poly(methyl vinyl ether/maleic anhydride); N-hydroxyethyliminodiacetic acid, nitrilotriacetic acid (NTA), N,N,N′,N′-ethylenediaminetetraacetic acid, N-hydroxyethyl-N, N′,N′-ethylenediaminetriacetic acid and N,N,N′,N″,N″-diethylenetriaminepentaacetic acid; α-hydroxy acids, such as citric acid, tartaric acid and gluconic acid; orthophosphates, such as trisodium phosphate, disodium phosphate, monosodium phosphate; condensed phosphates, such as sodium tripolyphosphate, tetrasodium pyrophosphate, sodium hexametaphosphate and sodium tetrapolyphosphate; 5-sulfo-8-hydroxyquinoline; and 3,5-disulfopyrocatechol, polycarboxylates, ethylene diamine tetraacetic acid (EDTA), diethylenetriaminepentaacetic acid (DTPA), N-hydroxyethyl-ethylenediamine-triacetic acid (HEDTA), ethylenediaminediacetate (EDDA), ethylenediaminedi (o-hydroxyphenylacetic) acid (EDDHA), cyclohexane diamine tetraacetic acid (CDTA), polyethyleneaminepolyacetic acids, lignosulfonate, Ca, K—, Na—, and ammonium lignosulfonates, fulvic acid, ulmic acid, nucleic acids, cyclodextrin, humic acid, pyrophosphate. However, those skilled in the art will appreciate that it is possible to utilize other chelating or complexing or sequestering agents without departing from the scope of the present invention. The chelating or complexing or sequestering agents are commercially manufactured and available through various companies.

According to an embodiment, the penetrant which is used in the composition include, but not limited to one or more of alcohol, glycol, glycol ether, ester, amine, alkanolamine, amine oxide, quaternary ammonium compound, triglyceride, fatty acid ester, fatty acid ether, N-methyl pyrrolidone, dimethyl formamide, dimethyl acetamide, or dimethyl sulfoxide, polyoxyethylenetrimethylolpropanemonooleate, polyoxyethylenetrimethylolpropanedioleate, polyoxyethylene trimethylol propane trioleate, polyoxyethylene sorbitan monooleate, polyoxyethylene sorbitol hexaoleate. However, those skilled in the art will appreciate that it is possible to utilize different penetrants without departing from the scope of the present invention. The penetrants are commercially manufactured and available through various companies.

According to an embodiment, the humectant is selected from, but not limited to one or more of polyoxyethylene/polyoxypropylene copolymers, particularly block copolymers, such as the Synperonic PE series of copolymers available from Uniqema or salts, derivatives thereof. Other humectants are propylene glycol, monoethylene glycol, hexylene glycol, butylene glycol, ethylene glycol, diethylene glycol, poly(ethylene glycol), poly(propylene glycol), glycerol and the like; polyhydric alcohol compounds such as propylene glycol ether, derivatives thereof. Also, the other humectants include aloe vera gel, alpha hydroxyl acids such as lactic acid, glyceryl triacetate, honey, lithium chloride, etc. The non-ionic surfactants mentioned above also act as humectants. However, those skilled in the art will appreciate that it is possible to utilize other conventionally known humectants without departing from the scope of the present invention. The humectants are commercially manufactured and available through various companies.

According to an embodiment, the humectant is present in the range of 0.1% to 90% w/w of the total composition. According to an embodiment, the humectant is present in the range of 0.1% to 70% w/w of the total composition. According to an embodiment, the humectant is present in the range of 0.1% to 60% w/w of the total composition. According to an embodiment, the humectant is present in the range of 0.1% to 50% w/w of the total composition. According to an embodiment, the humectant is present in the range of 0.1% to 30% w/w of the total composition. According to an embodiment, the humectant is present in the range of 0.1% to 10% w/w of the total composition.

The inventors have further determined that the composition of the present invention surprisingly has enhanced physical properties of suspensibility, improved viscosity, pourability, spontaneity of dispersion provides ease of handling and also reduces the loss of material while handling the product at the time of packaging as well as during field application.

Suspensibility is defined as the amount of active ingredient suspended after a given time in a column of liquid of stated height, expressed as a percentage of the amount of active ingredient in the original suspension. The test for suspensibility is done as per the CIPAC Handbook, “MT 184 Test for Suspensibility”.

According to an embodiment, the composition of the present invention has a suspensibility of at least 30%. According to an embodiment, the composition has a suspensibility of at least 40%. According to an embodiment, the composition has a suspensibility of at least 50%. According to an embodiment, the composition has a suspensibility of at least 60%. According to an embodiment, the composition has a suspensibility of at least 70%. According to an embodiment, the composition has a suspensibility of at least 80%. According to an embodiment, the composition has a suspensibility of at least 90%. According to an embodiment, the composition has a suspensibility of at least 99%. According to an embodiment, the pesticidal composition has a suspensibility of 100%.

According to an embodiment, the composition of the present invention demonstrates superior suspensibility under accelerated storage condition (ATS). According to an embodiment, the composition demonstrates a suspensibility of more than 90% under ATS. According to an embodiment, the composition demonstrates a suspensibility of more than 80% under ATS. According to an embodiment, the composition demonstrates a suspensibility of more than 70% under ATS. According to an embodiment, the composition demonstrates a suspensibility of more than 60% under ATS. According to an embodiment, the composition demonstrates a suspensibility of more than 50% under ATS. According to an embodiment, the composition demonstrates a suspensibility of more than 40% under ATS. According to an embodiment, the composition demonstrates a suspensibility of more than 30% under ATS.

According to an embodiment, the crop nutrition composition in the form of liquid suspension is not highly concentrated and is easily pourable. The viscosity of a fluid is a measure of its resistance to gradual deformation by shear stress or tensile stress.

According to an embodiment, viscosity of the liquid suspension is determined as per CIPAC MT-192. A sample is transferred to a standard measuring system. The measurement is carried out under different shear conditions and the apparent viscosities are determined. During the test, the temperature of the liquid is kept constant. According to an embodiment, the crop nutrition composition in the form of liquid suspension composition has a viscosity at 25° C. of about 10 cps to about 2000 cps which makes it pourable. According to an embodiment, the liquid suspension composition has viscosity at 25° C. of about 10 cps to about 1500 cps. According to an embodiment, the liquid suspension composition has viscosity at 25° C. of about 10 cps to about 1000 cps. According to an embodiment, the liquid suspension composition has viscosity at 25° C. of about 10 cps to about 500 cps.

According to an embodiment, the liquid suspension composition has a viscosity at 25° C. of about less than 2000 cps. According to an embodiment, the liquid suspension composition has a viscosity at 25° C. of about less than 1500 cps. According to an embodiment, the liquid suspension composition has a viscosity at 25° C. of about less than 500 cps. According to an embodiment, the liquid suspension composition has a viscosity at 25° C. of about 10 cps to about 400 cps. According to an embodiment, the liquid suspension composition has a viscosity at 25° C. of about 10 cps to about 300 cps. Too viscous and highly concentrated composition tends to form a cake making it unpourable and thus is undesirable.

According to an embodiment, the liquid suspension composition of the invention is easily pourable. The pourability is the measure of percent of residue.

According to an embodiment, the pourability of the composition is determined as per CIPAC MT-148.1 by allowing the composition to stand for 24 hours and the amount remaining in the container after a standardized pouring procedure is determined. The container is rinsed and the amount then remaining is determined and the maximum rinsed residue in percent is calculated. According to further embodiment, the pourability of composition is less than 5% rinsed residue. According to further embodiment, the pourability of the composition is preferably less than 2.5% rinsed residue. According to further embodiment, the pourability of the composition is more preferably less than 2.0% rinsed residue.

According to an embodiment, the spontaneity of dispersion is measured as per CIPAC MT 160. It involves preparing 250 ml of a mixture of formulation and water, mixed with only one inversion of the measuring cylinder. After standing under defined conditions the top nine-tenths is removed, and the remaining tenth assayed chemically, gravimetrically or by solvent extraction. The spontaneity of dispersion is readily calculated. According to an embodiment, the suspension concentrate composition has a spontaneity of dispersion of about 30%. According to an embodiment, the composition has a spontaneity of dispersion of about 40%. According to an embodiment, the composition has a spontaneity of dispersion of about 50%. According to an embodiment, the composition has a spontaneity of dispersion of about 60%. According to an embodiment, the composition has a spontaneity of dispersion of about 70%. According to an embodiment, the composition has a spontaneity of dispersion of about 80%. According to an embodiment, the composition has a spontaneity of dispersion of about 90%. According to an embodiment, the composition has a spontaneity of dispersion of about 95%. According to an embodiment, the composition has a spontaneity of dispersion of about 99%.

According to an embodiment, the dispersion stability of the oil dispersion and suspo-emulsion composition is measured as per CIPAC MT 180. A dispersion of prescribed concentration in water is prepared and aliquots are placed in two graduated emulsion tubes, which are then allowed to remain undisturbed for specified time in a upright and inverted positions at a constant temperature. The dispersion characteristics are observed immediately after the preparation of the dispersion, after a specified time, and after redispersion. According to an embodiment, the oil dispersion or suspo-emulsion composition disperses immediately. According to an embodiment, the oil dispersion or suspo-emulsion composition has dispersion stability for at least 30 min. According to an embodiment, the oil dispersion or suspo-emulsion composition has dispersion stability for at least 60 min. According to an embodiment, the oil dispersion or suspo-emulsion composition has dispersion stability for at least 24 hrs. The composition of the present invention is stable, disperses readily and even after 30 min and 24 hr without forming sediment or free oil layers and can be sprayed easily.

According to an embodiment, the present invention relates to a process for preparing a crop nutrition composition in the form of liquid suspension comprising a homogeneous mixture of one or more of water insoluble Magnesium salt, complex or derivative thereof and one or more of water insoluble Zinc salt, complex or derivative thereof and one or more of water insoluble Iron salt, complex or derivative thereof with at least one agrochemically acceptable excipient. The liquid suspension composition is in the form of a suspension concentrate, an oil dispersion or a suspo-emulsion composition.

According to further embodiment, the invention relates to a process for preparing a crop nutrition composition in the form of liquid suspension comprising a homogeneous mixture of one or more of water insoluble Magnesium salt, complex, or derivative thereof in the range of 1%-70% w/w of the total composition and one or more of water insoluble Zinc salt, complex or derivative thereof in the range of 1%-50% w/w of the total composition and one or more of water insoluble Iron salt, complex or derivative thereof in the range of 1%-50% w/w of the total composition with at least one agrochemically acceptable excipient; wherein elemental Iron is present in the range of 0.01% to 40% w/w of the total composition and wherein elemental Zinc is present in the range of 0.01% to 40% w/w of the total composition and wherein elemental Magnesium is present in the range of 0.01% to 50% w/w of the total composition and wherein the composition comprises of fine particles in the size range of 0.1 micron-20 microns.

According to an embodiment, the process of preparing the suspension concentrate composition involves homogenization of one or more of excipients for instance surfactants in water or water miscible solvents by feeding them into a vessel provided with stirring facilities. One or more of water insoluble Zinc salt, complex or derivative thereof, one or more of water insoluble Iron salt, complex or derivative thereof and one or more water insoluble Magnesium salt, complex or derivative thereof with at least one agrochemically acceptable excipient are further added to the homogenized blend and stirred continuously for about 5 to 10 minutes until the total mixture becomes homogeneous. Subsequently, the suspension obtained is passed through the wet mill to obtain a particle size in the range of 0.1 to 20 microns, preferably 0.1 to 10 microns. Then, requisite quantity of the structuring agent and optionally biocide or preservatives is added to the obtained suspension, under continuous homogenization.

According to an embodiment, the process of preparing an oil dispersion composition involves mixing of at least one water immiscible solvent, one or more surfactant to obtain a solvent-excipient mixture. The solvent mixture is optionally heated at 70-80° C. depending on the physical nature of the excipient. The solvent mixture is cooled down to room temperature. Further, adding one or more of water insoluble Zinc salt, complex or derivative thereof, one or more of water insoluble Iron salt, complex or derivative thereof and one or more water insoluble Magnesium salt, complex or derivative thereof and other optional agrochemical excipient to the solvent mixture to obtain a homogenized solution. Milling homogenized solution for about 15-30 minutes to obtain a dispersed medium of particle size in the range of 0.1-20 microns.

According to an embodiment, the process of preparing a suspo-emulsion composition comprising mixing or dissolving one or more of water insoluble Zinc salt, complex or derivative thereof, one or more of water insoluble Iron salt, complex or derivative thereof and one or more water insoluble Magnesium salt, complex or derivative thereof in an oil or a solvent and preparing a concentrated emulsion with required agrochemical excipients to obtain a first fraction. The process further comprises mixing an effective amount of surfactants or excipient to obtain a second fraction which is then milled to get the desired particle size. The two fractions obtained are then mixed using a suitable homogenizer for 30 minutes to obtain the suspoemulsion composition with the desired particle size of 0.1 to 50 microns. The two fractions obtained are then mixed in a mass mixer for 30 minutes to obtain the suspoemulsion composition with the desired particle size of 0.1 to 20 microns.

According to an embodiment, the invention further relates to the use of the crop nutrition composition in the form of liquid suspension as at least one of a nutrient composition, a crop strengthener composition, a soil conditioner composition, crop protection and a yield enhancer composition.

According to an embodiment, the invention further relates to a method for improving plant health or yield wherein the method comprises treating at least one of a plant, a plant propagation material, locus or parts, a seed, seedling; or surrounding soil with the liquid suspension composition of the present invention.

According to an embodiment, the invention also relates to a method of application of the crop nutrition composition in the form of liquid suspension comprising a homogeneous mixture of 1% to 50% by weight of one or more water insoluble Iron salt, complex or derivative thereof, 1% to 70% by weight of one or more water insoluble Magnesium salt, complex or derivative thereof, 1% to 50% by weight of one or more water insoluble Zinc salt, complex or derivative thereof and at least one agrochemically acceptable excipient, wherein elemental Iron is present in the range of 0.01% to 40% by weight and wherein elemental Zinc in the range of 0.01% to 40% by weight and wherein elemental Magnesium in the range of 0.01% to 50% by weight wherein the composition comprises of particles in the size range of 0.1 micron-20 microns and wherein the composition is applied to the seeds, seedlings, crops, a plant, plant propagation material, locus, plants parts or to the surrounding soil.

According to an embodiment, the present invention relates to a method of treating plants and meeting their nutritional requirement by making essential nutrients like Magnesium, Zinc and Iron available to them and also unlocking the other micronutrients and trace elements presents present in the soil and making them available to the plant which hitherto were not available because of various factors primarily being soil degradation on account of excessive use of synthetic fertilizers. The present invention also relates to strengthening the plants so as to withstand pest infestation. The present invention also relates to a method of biofortification of plant with essential micronutrients.

The present invention further provides balanced uptake of all nutrients, improves the crop health, improves the crop nutrition by facilitating the uptake of essential nutrients, protects the crop, enhances the crop yield, strengthens the plant or assists in conditioning the soil.

According to an embodiment, the present invention relates to a method for treating plants and meeting their nutritional requirement by enhancing uptake of Magnesium, Zinc and Iron by application of a composition comprising a homogeneous mixture of:
at least one water insoluble Iron salt, complex or derivative thereof in the range of 1-50% w/w of the total composition,
at least one water insoluble Zinc salt, complex or derivative thereof in the range of 1%-50% w/w of the total composition,
at least one water insoluble Magnesium salt, complex or derivative thereof in the range of 1%-70% w/w of the total composition,
at least one agrochemically acceptable excipient and
wherein elemental Iron is present in the range of 0.01% to 40% w/w of the total composition and
wherein elemental Zinc is present in the range of 0.01% to 40% w/w of the total composition and
wherein elemental Magnesium is present in the range of 0.01% to 50% w/w of the total composition,
wherein the composition comprises particles in the size range of 0.1 micron-20 microns.

The composition of the present invention further assists in regulating the soil pH so as to facilitate the balance uptake of micronutrients. Further, the composition of present invention was found to be effective independent of the soil pH making it a viable composition for all types of soils. More importantly, it was noted that the presence of Magnesium along with Zinc and Iron in the form of the composition of the present invention facilitated not only an uptake of significant proportion of Iron and Zinc present in the composition but also enabled plant uptake of micro nutrients like Boron, Manganese and Calcium etc entrapped in the soil.

The composition of the present invention was found to play a vital role in regulating soil pH and facilitating the uptake of nutrients even in soils which have been degraded or whose pH have been altered because of excessive use of synthetic fertilizers. The composition of the present invention met the nutritional need of plants by providing a balanced uptake of essential nutrients like Zinc, Iron and Magnesium, thus overcoming the challenge of providing a nutrient rich crop in calcareous soils which is known to provide an antagonism challenge for the uptake of these nutrients. It was further surprising to observe that the balanced uptake of nutrients leads to a healthier plant that could withstand pest infestation, a higher nutrient harvest in all soils types and finally improving the overall soil health. The present composition acts as a nutrient use efficient composition while meeting the need of crops by providing a multi nutritive solution with improved uptake by crops in a single application.

The present composition can be applied through a variety of methods. Methods of applying to the soil includes any suitable method, which ensures that the composition penetrates the soil, for example, nursery tray application, in furrow application, drip irrigation, sprinkler irrigation, soil drenching, soil injection, or incorporation into the soil, and such other methods. The composition also can be applied in the form of a foliar spray.

The rates of application or the dosage of the composition depends on the type of use, the type of crops, or the specific active ingredients in the composition but is such that the active ingredient, is in an effective amount to provide the desired action such as crop protection, crop yield and nutrient uptake.

### A. Preparation Examples

The following examples illustrate the basic methodology and versatility of the composition of the invention. The water insoluble sources of Iron, Magnesium and Zinc exemplified in the preparatory examples can be replaced by any other water insoluble salts, complexes or derivatives thereof of these nutrients as covered in the present invention varying the claimed concentration ranges respectively. It should be noted that this invention is not limited to these exemplifications.
1. Suspension Concentrate composition comprising 6% Zinc Borate, 9% Iron Fumarate and 9% Magnesium Carbonate.
   3 parts of polyacrylate copolymer and 4 parts of monoethylene glycol were added to water and homogenised by feeding them into a vessel provided with stirring facilities. 6 parts of Zinc Borate, 9 parts of Iron Fumarate and 9 parts of Magnesium Carbonate were further added to the homogenised blend and stirred continuously for approximately 10 minutes until the total mixture was homogeneous. To the above mixture, 1 part of Tensiofix RP, 2 parts of lignosulphonate was added. Subsequently, the suspension obtained was passed through the wet mill. Then, 0.1 part of xanthan gum, 0.1 part of benzisothiazoline and 0.2 part of polydimethylsiloxane emulsion were added under continuous homogenization to the resultant mixture, followed by sufficient amount of water to obtain the suspension concentrate composition. The composition had a particle size of D90 of 4 microns, viscosity of 550 cps and a suspensibility of 94%. The composition had a particle size of D90 of 3.6 microns, viscosity of 610 cps and a suspensibility of 90% under accelerated storage condition.
2. Suspension Concentrate composition comprising 3% Zinc Oxide, 30% Ferrous Carbonate and 1.75% Magnesium Carbonate
   The suspension concentrate composition was prepared as per Example 1 which comprises 3 parts of Zinc Oxide, 30 parts of Iron Phosphate, 1.75 parts of Magnesium Carbonate, 4 parts of monoethylene glycol, 3 parts of polyacrylate copolymer, 1 part of Naphthalenesulphonate condensate sodium salt, 2 parts of lignin sulfonate, 0.2 parts of silicone defoamer, 0.1 parts of M Z36, 0.1 part of xanthan gum and sufficient quantity of water to make 100%.
   The composition had a particle size of D90 of 5.2 microns, viscosity of 300 cps and suspensibility of 88%. The composition exhibited a particle size of D90 of 5.8 microns, viscosity of 450 cps and suspensibility of 80% under accelerated storage condition.
3. Suspension Concentrate composition comprising 17.5% Zinc Borate, 10.5% Ferric Oxide and 17.5% Magnesium Silicate.
   The suspension concentrate composition was prepared as per Example 1 which comprises 17.5 parts of Zinc Borate, 10.5 parts of Ferric Oxide, 17.5 parts of Magnesium Silicate, 4 parts of monoethylene glycol, 3 parts of polyacrylate graft copolymer, 1 part of Lignosulphonate, 2 parts of naphthalene sulfonate condensate, 0.2 parts of silicone defoamer, 0.1 parts of benzisothiazolinone, 0.5 part of Carboxymethyl cellulose sodium and water to make 100% composition.
   The composition had a particle size of D90 of 8.6 microns, viscosity of 720 cps and suspensibility of 89%. The composition exhibited a particle size of D90 of 3.7 microns, viscosity of 1000 cps and suspensibility of 83% under accelerated storage condition.
4. Suspension Concentrate composition comprising 8% Zinc Oxide, 4% Iron (II) Oxide and 20% Magnesium Silicate Hydrate.
   The suspension concentrate composition was prepared as per Example 1 which comprises 8 parts of Zinc Oxide, 4 parts of Iron (II) Oxide, 20 parts of Magnesium Silicate Hydrate, 4 parts of monoethylene glycol, 3 parts of alkylnaphthalene sulphonate condensate sodium salt, 1 part of Disperbyk 2010, 2 parts of lignin sulfonate, 0.2 parts of petroleum oil based defoamer, 0.1 parts of benzisothiazolinone, 0.1 part of xanthan gum and sufficient quantity of water to make 100% composition.
   The composition had a particle size (D90) of 2.7 microns, viscosity of 1000 cps and suspensibility of 94%. The composition exhibited a particle size (D90) of 8.1 microns, viscosity of 1300 cps and suspensibility of 90% under accelerated storage condition.
5. Suspension Concentrate composition comprising 5% Zinc Phosphate, 5% Iron (II) Fumarate and 15% Magnesium Hydroxide.
   The suspension concentrate composition was prepared as per Example 1 which comprises 5 parts of Zinc Phosphate, 5 parts of Iron Fumarate, 15 parts of Magnesium Hydroxide, 4 parts of propylene glycol, 3 parts of acid resin copolymer, 3 parts of acrylic graft copolymer, 0.2 parts of polydimethylsiloxane defoamer, 0.1 parts of benzothiazolinone, 0.1 part of xanthan gum, 64.6 parts of water.
   The composition had a particle size of D90 of 3.0 microns, viscosity of 1000 cps and suspensibility of 91%. The composition exhibited a particle size of D90 of 8.8 microns, viscosity of 1200 cps and suspensibility of 87% under accelerated storage condition.
6. Suspension Concentrate composition comprising 6% Zinc Oxide, 6% Ferric Oxide and 12% Magnesium Oxide.
   The suspension concentrate composition was prepared as per Example 1 which comprises 6 parts of Zinc Oxide, 6 parts of Ferric Oxide, 12 parts of Magnesium Oxide, 4 parts of monoethylene glycol, 3 parts of polyacrylate graft copolymer, 3 parts of Geropon SC213, 0.2 parts of silicone defoamer, 0.1 parts of benzisothiazolinone, 0.1 part of xanthan gum and water to make 100% composition. The composition had a particle size of D90 of 4.6 microns, viscosity of 750 cps and suspensibility of 89%. The particle size of D90 was 9 microns, viscosity was 790 cps and suspensibility was 80% under accelerated storage condition.
7. Suspension Concentrate composition comprising 2.5% Zinc Oxide, 5% Iron (III) Phosphate and 40% Magnesium Carbonate in monoethylene glycol.
   The suspension concentrate composition was prepared by dispersing 2.5 parts of Zinc oxide, 5 parts of Iron (III) Phosphate and 40 parts of Magnesium Carbonate in 33 parts of monoethylene glycol (as solvent). 23 parts of polyoxyethylene alkyl ether, 2.5 gms of Sodium ligno sulphonate, 3 parts of polyacrylate, were added to this slurry which was then passed through a suitable wet mill. 1 part of fumed anhydrous silica was added under stirring and the stirring is continued for 30 minutes.
   The composition had a particle size of D90 of 3.2 microns, viscosity of 200 cps and suspensibility of 86%. The particle size of D90 was 4 microns, viscosity was 300 cps and suspensibility was 78% after ATS.
8. Oil dispersion composition comprising 10% Zinc Oxide, 5% Ferrous carbonate and 15% Magnesium Oxide.
   4 parts of non-ionic block copolymer was mixed homogenously with 54 parts of sunflower seed oil. 10 parts of Zinc Oxide, 5 parts of Ferrous carbonate and 15 parts of Magnesium Oxide were added to sunflower oil The blend obtained was further mixed with 10 parts of graft polymer in suitable mixing equipment and milled to form a slurry or wet mix. 2 parts of hydrophobically modified silica was added to the slurry to obtain a stable homogenous oil dispersion.
   The composition had the following particle size of D90 of 3.5 microns. The composition had a suspensibility of 95%, pourability of 0.5% residue, viscosity of 350 cps. The composition further demonstrated suspensibility of about 91%, particle size of D90 of 4 microns, pourability of 1% residue and viscosity of 450 cps under accelerated storage condition.
9. Suspension Concentrate composition comprising 3.75% Zinc Carbonate, 3.75% Iron Silicate and 7.5% Magnesium Oxide.
   The suspension concentrate composition was prepared as per Example 1 which comprises 3.75 parts of Zinc Carbonate, 3.75 parts of Iron Silicate and 7.5 parts of Magnesium Oxide, 4 parts of glycerol, 3 parts of 1 part of sodium lauryl sulphate, 2 parts of polyacrylate graft copolymer, 3 parts of Kraft lignin, 0.2 parts oil based defoamer and water to make 100%.
   The composition had a particle size (D90) of 4.2 microns, viscosity of 580 cps and suspensibility of 86%. The composition exhibited a particle size of (D90) 4.8 microns, viscosity of 650 cps and suspensibility of 80% after ATS.
10. Suspo-emulsion composition comprising 2.5% of Zinc Phosphate, 5% of Iron Silicate and 12.5% of Magnesium Hydroxide.
   The suspoemulsion composition was prepared which comprises 2.5 parts of Zinc Phosphate, 5 parts of Iron silicate, 12.5 parts of Magnesium Hydroxide, 6 parts of propylene glycol, 30 parts of water, 3 parts of acid resin copolymer, 3 parts of acrylic graft copolymer, 0.2 parts of polydimethylsiloxane defoamer, Subsequently, the suspension obtained was passed through the wet mill. Then, 0.1 part of xanthan gum, 0.1 part of benzisothiazoline and 0.2 part of polydimethylsiloxane emulsion and 10 parts of sunflower oil with balance quantity of water to under continuous homogenization to obtain the suspoemulsion. The composition had a particle size of D90 of 3.0 microns, viscosity of 800 cps and suspensibility of 80%. The composition exhibited a particle size of D90 of 4.8 microns, viscosity of 1200 cps and suspensibility of 78% under accelerated storage condition.

### B. Field Study

Experiment 1: To study the impact of Liquid suspension composition comprising water insoluble Zinc salt and water insoluble Magnesium salt and water insoluble Iron salt on Soyabean crop.

### Field Experiment Methodology:

The field trial was carried out to see the effect of Liquid suspension composition comprising water insoluble Zinc salt and water insoluble Magnesium salt and water insoluble Iron salt in Soyabean at Indore, Madhya Pradesh. The trial was laid out during Kharif season in Randomized Block Design (RBD) with ten treatments including untreated control, replicated four times. For each treatment, plot size of 30 sq·m (6 m×5 m) was maintained. The test product compounds various Zinc salts, Iron salts, Magnesium salts alone and their combination in liquid suspension composition as per the present invention varying concentration range with prescribed dose were applied to the soil at the time of sowing. The Soybean crop in trial field was raised following good agricultural practices.

Details of Experiment:

| a) Trial Location | Indore, Madhya Pradesh |
|---|---|
| b) Crop & Variety | Soybean (JS335) |
| c) Experiment season | Kharif 2022 |
| d) Trial Design | Randomized Block Design |
| e) Replications | 4 |
| f) Treatment | 10 |
| g) Plot size | 6 m × 5 m = 30 sq. m |
| h) Date of sowing | 7 Jul. 2022 |
| i) Date of Application | 7 Jul. 2022 |
| j) Method of application | Soil application |
| k) Date of Harvesting | 10 Oct. 2022 |
| l) Soil pH | 7-7.5 |

The observations were recorded at the harvesting time and the mean data was presented in Table 1 to enumerate the efficacy of the liquid suspension composition prepared as per the embodiment of the present invention.

TABLE 1

| | Formulation | Dose of nutrient salt in | | | Yield of | | Expected |
|---|---|---|---|---|---|---|---|
| | dosage in | g/ha | | | Soybean | % Increase | % increase |
| Compositions | g/ha | Zinc | Iron | Magnesium | Kg/ha | in Yield | in Yield |
| T1- Zinc | 400 | 17.30 | | | 1100 | 22.22 | 49.07 |
| Carbonate | | | | | | | (1.61*) |
| 15% | | | | | | | |
| (elemental | | | | | | | |
| Zinc 4.32%) | | | | | | | |
| SC | | | | | | | |
| T2- Iron | 400 | 0.00 | 19.72 | | 1020 | 13.33 | |
| Silicate 15% | | | | | | | |
| (elemental | | | | | | | |
| Iron 4.928%) | | | | | | | |
| SC | | | | | | | |
| T3- | 400 | 0.00 | 0.00 | 72.3 | 1120 | 24.44 | |
| Magnesium | | | | | | | |
| Oxide 30% | | | | | | | |
| (elemental | | | | | | | |
| Magnesium | | | | | | | |
| 18.09%) SC | | | | | | | |
| T4- Zinc | 400 | 17.3 | 19.72 | 72.3 | 1610.00 | 78.89 | |
| Carbonate | | | | | | | |
| 15% | | | | | | | |
| (elemental | | | | | | | |
| Zinc 4.32%) + | | | | | | | |
| Iron Silicate | | | | | | | |
| 15% | | | | | | | |
| (elemental | | | | | | | |
| Iron 4.928%) + | | | | | | | |
| Magnesium | | | | | | | |
| Oxide 30% | | | | | | | |
| (elemental | | | | | | | |
| Magnesium | | | | | | | |
| 18.09%)-SC | | | | | | | |
| T5- Zinc | 500 | 29.3 | | | 1105 | 22.78 | 55.48 |
| Silicate 10% | | | | | | | (1.34*) |
| (elemental | | | | | | | |
| Zinc 5.86%) | | | | | | | |
| SC | | | | | | | |
| T6- Iron (II) | [—\00.0 | | 32.8 | | 1103 | 22.56 | |
| Fumarate | | | | | | | |
| (elemental | | | | | | | |
| Iron 6.57%) | | | | | | | |
| 20% SC | | | | | | | |
| T7- | 500.0 | | | 50.47 | 1130 | 25.56 | |
| Magnesium | | | | | | | |
| Carbonate | | | | | | | |
| (elemental | | | | | | | |
| Magnesium | | | | | | | |
| 10.09%) 35% | | | | | | | |
| SC | | | | | | | |
| T8- Zinc | 500 | 29.3 | 32.87 | 50.47 | 1570 | 74.44 | |
| Silicate 10% | | | | | | | |
| (elemental | | | | | | | |
| Zinc 5.86%) + | | | | | | | |
| Iron (II) | | | | | | | |
| Fumarate 20% | | | | | | | |
| (elemental | | | | | | | |
| Iron 6.57%) + | | | | | | | |
| Magnesium | | | | | | | |
| Carbonate | | | | | | | |
| 35% | | | | | | | |
| (elemental | | | | | | | |
| Magnesium | | | | | | | |
| 10.09%) -SC | | | | | | | |
| T9- Zinc | 500 | 40.17 | | 120.65 | 1145 | 27.22 | |
| Oxide 10% | | | | | | | |
| (elemental | | | | | | | |
| Zinc 8.03%) + | | | | | | | |
| Magnesium | | | | | | | |
| Oxide 40% | | | | | | | |
| (elemental | | | | | | | |
| Magnesium | | | | | | | |
| 24.13%) SC | | | | | | | |
| composition | | | | | | | |
| as per prior art | | | | | | | |
| CN113214010 | | | | | | | |
| T10-Untreated | | | | | 900 | | |

| | Nutrient | | | | | |
|---|---|---|---|---|---|---|
| | concentration | | | Number of | Mean Plant | Number of |
| | (mg/100 g of seeds) | | | pods/ | Height (cm) | branches/ |
| Compositions | Zn | Fe | Mg | plant | 30 DAA | plant |
| T1- Zinc | 2.5 | 1 | 63 | 13.7 | 41.20 | 4.1 |
| Carbonate 15% | | | | | | |
| (elemental Zinc | | | | | | |
| 4.32%) SC | | | | | | |
| T2- Iron Silicate | 1.2 | 2.1 | 62 | 12.8 | 39.30 | 3.4 |
| 15% (elemental | | | | | | |
| Iron 4.928%) | | | | | | |
| SC | | | | | | |
| T3- Magnesium | 1.3 | 1.1 | 115 | 13.5 | 38.10 | 2.9 |
| Oxide 30% | | | | | | |
| (elemental | | | | | | |
| Magnesium | | | | | | |
| 18.09%) SC | | | | | | |
| T4- Zinc | 5.8 | 4.7 | 240 | 18.9 | 46.30 | 7.9 |
| Carbonate 15% | | | | | | |
| (elemental Zinc | | | | | | |
| 4.32) + Iron | | | | | | |
| Silicate 15% | | | | | | |
| (elemental Iron | | | | | | |
| 4.928%) + | | | | | | |
| Magnesium | | | | | | |
| Oxide 30% | | | | | | |
| (elemental | | | | | | |
| Magnesium | | | | | | |
| 18.09%)-SC | | | | | | |
| T5- Zinc | 2.1 | 1.2 | 67 | 12.9 | 39.10 | 3.1 |
| Silicate 10% | | | | | | |
| (elemental Zinc | | | | | | |
| 5.86%) SC | | | | | | |
| T6- Iron (II) | 1.4 | 2.3 | 65 | 13.7 | 38.20 | 4.2 |
| Fumarate | | | | | | |
| (elemental Iron | | | | | | |
| 6.57%) 20% SC | | | | | | |
| T7- Magnesium | 1.5 | 1.2 | 189 | 13.2 | 39.20 | 5.2 |
| Carbonate | | | | | | |
| (elemental | | | | | | |
| Magnesium | | | | | | |
| 10.09%) 35% | | | | | | |
| SC | | | | | | |
| T8- Zinc | 6.58 | 4.1 | 250 | 18.4 | 45.30 | 8.6 |
| Silicate10% | | | | | | |
| (elemental Zinc | | | | | | |
| 5.86%) + Iron | | | | | | |
| (II) Fumarate | | | | | | |
| 20% (elemental | | | | | | |
| Iron 6.57%) + | | | | | | |
| Magnesium | | | | | | |
| Carbonate 35% | | | | | | |
| (elemental | | | | | | |
| Magnesium | | | | | | |
| 10.09%) -SC | | | | | | |
| T9- Zinc Oxide | 2.3 | 0.9 | 195 | 13.5 | 38.14 | 5.1 |
| 10% (elemental | | | | | | |
| Zinc 8.03%) + | | | | | | |
| Magnesium | | | | | | |
| Oxide 40% | | | | | | |
| (elemental | | | | | | |
| Magnesium | | | | | | |
| 24.13%) | | | | | | |
| composition as | | | | | | |
| per prior art | | | | | | |
| CN113214010 | | | | | | |
| T10-Untreated | 1.1 | 0.2 | 10.8 | 12.2 | 37.00 | 2.5 |

*Synergy factor
*DAA Days after Application
*The water insoluble Magnesium, Iron and Zinc salts selected and the concentration range of these nutrients covered in above table are exemplary and can be replaced with other water insoluble Magnesium, Iron and Zinc salts as per the embodiment of the present invention.

From the data observed in the Table 1, it can be seen that the compositions T4 and T8 as per the embodiments of the present invention demonstrate a synergistic behavior.

“Synergy” is as defined by Colby S. R. in an article entitled “Calculation of the synergistic and antagonistic responses of herbicide combinations” published in Weeds, 1967, 15, p. 20-22. The action expected for a given combination of two active components can be calculated as follows:

$$E = X + Y + Z - ( X Y + Y Z + X Z ) / 100 + ( X Y Z / 10000 )$$

Where,
E=Expected % effect by mixture of two products X, Y and Z in a defined dose.
X=Observed % effect by product A
Y=Observed % effect by product B
Z=Observed % effect by product C

The synergy factor (SF) is calculated by Abbott's formula (Eq. (2) (Abbott, 1925). SF=Observed effect/Expected effect

Where, SF>1 for Synergistic reaction; SF<1 for antagonistic reaction; SF=1 for additive reaction.

When the percentage of yield effect observed for the combination is greater than the expected percentage, synergistic effect of the combination can be inferred. When the percentage of yield effect observed for the combination is equal to the expected percentage, merely an additive effect may be inferred and wherein the percentage of yield effect observed for the combination is lower than the expected percentage, an antagonistic effect of the combinations can be inferred.

It can be observed that the synergy factor is 1.41 and 1.10 for treatments T4 and T8 as seen from Table 1 which depicts that the SC compositions of “Zinc Carbonate+Iron Silicate+Magnesium Oxide”, “Zinc Silicate+Iron (II) Fumarate+Magnesium Carbonate” respectively are synergistic in nature. This synergistic behavior of “water insoluble Zinc salt and water insoluble Magnesium salt and water insoluble Iron salt” in the form of SC as per embodiment of the present invention can be observed from the yield of Soybean crop. The four treatments namely T1 (15% Zinc Carbonate SC), T2 (15% Iron Silicate SC), T3 (30% Magnesium Oxide SC) and T4 [Zinc Carbonate 15% (elemental Zinc 4.32% SC)+Iron Silicate 15% (elemental Iron 4.928% SC)+Magnesium Oxide 30% (elemental Magnesium 18.09%)] SC were applied at active dosage i. e. 17.3 g/ha of Zinc, 19.72 g/ha of Iron and 72.39 g/ha of Magnesium. Treatment T4 exhibits highest yield of about 1610 Kg/ha when compared to treatment T1 with a yield—1100 Kg/ha, T2 with a yield—1020 K g/ha and T3 with a yield—1120 Kg/ha. The expected % increase in yield was 49.07% but the observed % increase in yield for Treatment T4 was 78.89%, demonstrating synergistic effect.

Thus, the combination of Zinc Carbonate 15%+Iron Silicate 15%+Magnesium Oxide 30% in SC form as per embodiment of the present invention is synergistic and provides higher crop yield as compared to the application of individual actives when applied at the same dosage. Similar trends in terms of yield were also observed with the treatments T8, when compared to treatments T5, T6, T7 respectively which depicts the synergistic behavior of the compositions as per the embodiment of the present invention.

It can be appreciated from the observed results that plant height and number of branches in Soybean crop were higher in treatment T4 with Zinc Carbonate 15%+Iron Silicate 15%+Magnesium Oxide 30%—SC—as compared to the individual applications of actives. On comparing treatments T5-T8 it can be noted that Treatment T8 has plant height and number of branches of 45.30 cm and 8.6 respectively whereas treatment T5, T6 and T7 has a plant height of 39.10, 38.20, 39.20 cm, and 3.1, 4.2, and 5.2 branches respectively.

The untreated control also has a plant height of 37 cm, and 2.5 branches. It was also observed that the leaves of soybean plot treated with treatments T4 and T8 were greener as compared to Treatments T1-T3, T5-T7 and the untreated plot where yellow leaves were observed.

From Table 1 it can also be observed that availability of Zinc, Magnesium and Iron with respect to SC composition prepared according to an embodiment of the present invention is greater than those observed with the same actives applied stand alone at soil pH 7-7.5. It can be also seen that 6.58 mg, 4.1 mg and 250 mg of Zinc, Iron and Magnesium were available for uptake with respect to the SC composition of T8-Zinc Silicate 10% (elemental Zinc 5.86%)+Iron (II) Fumarate 20% (elemental Zinc 6.57%)+Magnesium Carbonate 35% (elemental Magnesium 10.09%)—SC whereas only 2.1 mg, 1.4 mg, 1.5 mg of Zinc, 1.2 mg, 2.3 mg, 1.2 mg of Iron and 67 mg, 65 mg, 189 mg of Magnesium were available for uptake to the plants by application of treatments T5, T6 and T7 of the individual actives respectively.

This appreciable increase in the availability of Zinc and Iron observed in Treatment T4 and T8 was noted to be on account of the presence of Magnesium along with Zinc and Iron in the composition formulated as per the embodiment of the invention i.e., in the form of liquid suspension with particle size in the range of 0.1 microns to 20 microns which facilitated the increased availability of the entire range of micronutrients present in the composition i.e., Magnesium, Iron and Zinc for uptake by the crops.

It was thus noted that the composition comprising a combination of water insoluble Zinc salt and water insoluble Magnesium salt and water insoluble Iron salt in the form of liquid suspension demonstrates a better uptake of Magnesium, Zinc and Iron when compared to an individual application of the said actives as well as an application of a composition of only Iron and Zinc which is devoid of Magnesium.

Moreover, when compared to treatment T9 (the composition prepared as per the teachings of prior art), treatment T8 prepared as per the embodiment of the present invention also depicted enhanced yield despite being applied at a reduced dosage of Zinc and Magnesium. Further, it can be also seen that 2.3 mg, 0.9 mg and 195 mg of Zinc, Iron and Magnesium were available for uptake with respect to the SC composition of T9—Zinc Oxide 10% (elemental Zinc 8.03%)+Magnesium Oxide 40% (elemental Magnesium 24.13%)—a prior art composition whereas 6.58 mg, 4.1 mg, 250 mg of Zinc, Iron and Magnesium were available for uptake to the plants with respect to Treatment T8. Thus, even when applied at higher dosages of actives i.e. Zinc and Magnesium and using same formulation type, treatment T9 shows less uptake of these nutrients. On the contrary, treatment T8 shows much higher uptake of same nutrients. This appreciable increase in the availability of nutrients observed in Treatment T8 vis-à-vis T9 was noted to be on account of nature of the composition formulated as per the embodiment of the invention i.e., in the form of liquid suspension with particle size in the range of 0.1 microns to 20 microns which facilitated the increased availability of the entire range of micronutrients present in the composition i.e., Magnesium, Iron and Zinc for uptake by the crops.

From the aforementioned data, it can be concluded that the composition comprising of “water insoluble Zinc salt and water insoluble Magnesium salt and water insoluble Iron salt” in the form of SC as per the embodiment of the present invention at different dosages and at claimed concentration ranges demonstrated significantly higher uptake of micronutrients, higher yield, plant height, root development and number of branches.

The inventors of the present invention have further observed that apart from the Zinc, Magnesium and Iron salts listed in the Table 1 above, other Zinc, Magnesium and Iron Salts as claimed in the present application also exhibited similar effect when applied as per the embodiment of the present invention.

### Experiment 2: To Study the Effect of Liquid Suspension Composition of Present Invention on Tomato Crop:

Field trial was conducted for the evaluation of an embodiment of the composition of the present invention at Chakan, Pune, Maharashtra on Tomato crop, variety: Abhinav. The trials were laid down in Randomized Block Design (RBD) with nine treatments including untreated control, replicated four times. For each treatment, plot size of 30 sq·m (6 m×5 m) was maintained. The test nutritional compositions various Zinc salts, Iron salts, Magnesium salts alone and their combination in suspension concentrate as per the present invention varying concentration range with prescribed dose were applied as basal application at the time of sowing of Tomato crop.

The details of the experiment are as follows:

| a) Trial Location | Chakan, Pune |
|---|---|
| b) Crop & Variety | Abhinav (Syngenta) |
| c) Experiment season | Kharif 2022 |
| d) Trial Design | Randomized Block Design |
| e) Replications | 4 |
| f) Treatment | 9 |
| g) Plot size | 6 m × 5 m = 30 sq. m |
| h) Date of sowing | 15 Jul. 2022 |
| i) Date of Application | 15 Jul. 2022 |
| j) Method of application | Soil application |
| k) Date of Harvesting | 3 Nov. 2022 |
| l) Soil pH | 7-7.5 |

The observations were recorded at the harvesting time and the mean data was presented in Table 2 to enumerate the efficacy of the liquid suspension of “water insoluble Zinc salt and water insoluble Magnesium salt and water insoluble Iron salt” prepared as per the embodiment of the present invention.

TABLE 2

| | | | | | | | Expected | Fruit |
|---|---|---|---|---|---|---|---|---|
| | | | | | Fruit | % | % | weight |
| | Formulation | Dosage of nutrients | | | yield | Increase | Increase | (Avg of |
| | dosage | g/ha | | | (avg of 3 | in | in | 5 fruits) |
| Treatment Details | in g/ha | Zinc | Iron | Magnesium | picking) | Yield | yield | (g) |
| T1- Zinc Oxide 40% | 400.0 | 128.54 | | | 10000 | 13.64 | 21.68 | 310 |
| (elemental Zinc | | | | | | | | |
| 32.13%) SC | | | | | | | | |
| T2- Iron (III) Phosphate | 400.0 | 0.00 | 29.62 | | 9200 | 4.55 | | 290 |
| 20% (elemental Iron | | | | | | | | |
| 7.406%) SC | | | | | | | | |
| T3- Magnesium | 400.0 | 0.00 | 0.00 | 5.77 | 9240 | 5.00 | | 300 |
| Carbonate5% | | | | | | | | |
| (elemental Magnesium | | | | | | | | |
| 1.44%) SC | | | | | | | | |
| T4- Zinc Oxide 40% | 400.0 | 128.54 | 29.62 | 5.77 | 14600 | 65.91 | | 455 |
| (elemental Zinc | | | | | | | | |
| 32.13%) + Iron (III) | | | | | | | | |
| Phosphate 20% | | | | | | | | |
| (elemental Iron | | | | | | | | |
| 7.406%) + Magnesium | | | | | | | | |
| Carbonate 5% | | | | | | | | |
| (elemental Magnesium | | | | | | | | |
| 1.44%) SC | | | | | | | | |
| T5- Zinc Silicate 10% | 500.0 | 29.34 | | | 9240 | 5.00 | 31.30 | 280 |
| (elemental Zinc 5.86%) | | | | | | | | |
| SC | | | | | | | | |
| T6- Iron Fumarate 25% | 500.0 | 0.00 | 41.09 | | 9600 | 9.09 | | 300 |
| (elemental Iron | | | | | | | | |
| 9.861%) SC | | | | | | | | |
| T7- Magnesium | 500.0 | 0.00 | 0.00 | 62.54 | 10600 | 20.45 | | 310 |
| Hydroxide 30% | | | | | | | | |
| (elemental Magnesium | | | | | | | | |
| 12.502%) SC | | | | | | | | |
| T8- Zinc Silicate 10% | 500.0 | 29.34 | 41.09 | 62.54 | 15800 | 79.55 | | 490 |
| (elemental Zinc 5.86%) + | | | | | | | | |
| Iron Fumarate 25% | | | | | | | | |
| (elemental Iron | | | | | | | | |
| 9.861%) + Magnesium | | | | | | | | |
| Hydroxide 30% | | | | | | | | |
| (elemental Magnesium | | | | | | | | |
| 12.502%) -SC | | | | | | | | |
| T9-Untreated | | | | | 8800 | | | 270 |

From the data observed in the Table 2, it can be seen that the compositions T4 and T8 as per the embodiments of the present invention demonstrate a synergistic behavior.

Based on the data presented in Table 2 and the calculations made, the expected percentage increase in the groundnut kernel yield was found to be 21.68% and 31.30%. However, it can be clearly seen from the Table 2 above that the treatment T4 with Zinc Oxide 40% (elemental Zinc 32.13%)+Iron (III) Phosphate 20% (elemental Iron 7.406%)+Magnesium Carbonate 5% (elemental Magnesium 1.44%) SC as per the embodiment of the present invention showed a yield increase of 65.91% of Tomato fruit and treatment T8 with Zinc Silicate 10%+Iron Fumarate 25%+Magnesium Hydroxide 30%—SC composition, as per the embodiment of the present invention showed a yield increase of 79.55% of Tomato fruit.

However, treatments T1 with 40% Zinc Oxide SC, T2 with 20% Iron (III) Phosphate SC and T3 with 5% Magnesium Carbonate SC demonstrated only a 13.64%, 4.55% and 5% increase in the fruit of Tomato respectively. Similarly, treatments T5 with 10% Zinc Silicate SC, T6 with 25% Iron Fumarate SC and T7 with 30% Magnesium Hydroxide SC demonstrated only a 5%, 9.09% and 20.45% increase in the fruit yield of Tomato crop respectively. Thus, the treatments T4 and T8 with liquid suspension as per the embodiments of the present invention demonstrated a synergistic effect, as compared to the treatment with individual actives. The results are all the more surprising as the treatments T1-T4 and T5-T8 were applied at same dosage of Zinc salt, Iron salt and Magnesium salt being applied to the soil i.e. 128.54 g/ha of Zinc, 29.62 g/ha of Iron, 5.77 g/ha of Magnesium and 29.34 g/ha of Zinc, 41.09 g/ha of Iron, 62.54 g/ha of Magnesium respectively.

Further, Treatments T4 and T8 exhibited highest fruit weight when compared to fruit weights observed for treatments T1-T7.

From the aforementioned data, it can be concluded that the composition comprising of “water insoluble Zinc salt and water insoluble Magnesium salt and water insoluble Iron salt” in the form of SC as per the embodiment of the present invention at different dosages and at claimed concentration ranges demonstrated significantly higher yield, fruit weight.

The inventors of the present invention have further observed that apart from the Zinc, Magnesium and Iron salts listed in the Table 2 above, other Zinc, Magnesium and Iron Salts as claimed in the present application also exhibited similar effect when applied as per the embodiment of the present invention.

### Experiment No 3: To Assess the Impact of Particle Size Distribution in the Composition Comprising Zinc Oxide+Ferric Oxide+Magnesium Oxide—SC on Yield of Brinjal

### Field Experiment Methodology:

The field trials were carried out to observe the effect of different ranges of particle size with regard to the composition of Zinc Oxide+Ferric Oxide+Magnesium Oxide—SC on the yield of Brinjal at West Bengal.

The trial was laid out during spring season in Randomized Block Design (RBD) with eight treatments including untreated control, replicated four times. For each treatment, plot size of 40 sq·m (8 m×5 m) was maintained. The Brinjal crop in trial field was raised following good agricultural practice.

| a) Trial Location | “North 24 parganas, West bengal” |
|---|---|
| b) Crop & Variety | Brinjal (VNR212) |
| c) Experiment season | Kharif 2022 |
| d) Trial Design | Randomized Block Design |
| e) Replications | 4 |
| f) Treatment | 8 |
| g) Plot size | 8 m × 5 m = 30 sq. m |
| h) Date of sowing | 24 Aug. 2022 |
| i) Date of Application | 24 Aug. 2022 |
| j) Method of application | Soil application |
| k) Date of Harvesting | 10 Dec. 2022 |
| l) Soil pH | 7-7.5 |

The observations on yield were recorded at the time of harvesting and the mean data was presented in table 3 to see the impact of particle size distribution of the SC composition comprising “Water insoluble Zinc salt and Water insoluble Magnesium salt and water insoluble Iron salt” on yield of Brinjal.

TABLE 3

| | | | | | | | | Yield | |
|---|---|---|---|---|---|---|---|---|---|
| | | | | | Nutrient uptake | | | kg/ha | |
| | Particle | Dosage of active | | | (mg/100 gm of | | | (Avg of | % |
| | size | content (g/ha) | | | fruit) | | | two | Increase |
| Compositions | (micron) | Zn | Fe | Mg | Zn | Fe | Mg | pickings) | in Yield |
| T1- Zinc | 0.1-20 | 24.10 | 20.98 | 36.15 | 5.00 | 4.10 | 170.00 | 3500 | 84.21 |
| Oxide 6% | | | | | | | | | |
| (elemental | | | | | | | | | |
| Zinc 4.82%) + | | | | | | | | | |
| Ferric | | | | | | | | | |
| Oxide 6% | | | | | | | | | |
| (elemental | | | | | | | | | |
| Iron 4.196%) + | | | | | | | | | |
| Magnesium | | | | | | | | | |
| Oxide 12% | | | | | | | | | |
| (elemental | | | | | | | | | |
| Magnesium | | | | | | | | | |
| 7.23%)-SC % | | | | | | | | | |
| @ 500 g/ha | | | | | | | | | |
| T2- Zinc | 0.1-50 | 24.10 | 20.98 | 36.18 | 2.9 | 1.9 | 105 | 2670 | 40.53 |
| Oxide 6% | | | | | | | | | |
| (elemental | | | | | | | | | |
| Zinc 4.82%) + | | | | | | | | | |
| Ferric | | | | | | | | | |
| Oxide 6% | | | | | | | | | |
| (elemental | | | | | | | | | |
| Iron 4.196%) + | | | | | | | | | |
| Magnesium | | | | | | | | | |
| Oxide 12% | | | | | | | | | |
| (elemental | | | | | | | | | |
| Magnesium | | | | | | | | | |
| 7.23%) SC % | | | | | | | | | |
| @ 500 g/ha | | | | | | | | | |
| T3- Zinc | 21-50 | 24.10 | 20.98 | 36.18 | 1.9 | 1.7 | 85 | 2600 | 36.84 |
| Oxide 6% | | | | | | | | | |
| (elemental | | | | | | | | | |
| Zinc 4.82%) + | | | | | | | | | |
| Ferric | | | | | | | | | |
| Oxide % | | | | | | | | | |
| (elemental | | | | | | | | | |
| Iron 4.196%) | | | | | | | | | |
| Magnesium | | | | | | | | | |
| Oxide 12% | | | | | | | | | |
| (elemental | | | | | | | | | |
| Magnesium | | | | | | | | | |
| 7.23%)-SC % | | | | | | | | | |
| @ 500 g/ha | | | | | | | | | |
| T4- Zinc | 50-100 | 24.10 | 20.98 | 36.18 | 2.2 | 1.5 | 75.00 | 2570 | 35.26 |
| Oxide 6% | | | | | | | | | |
| (elemental | | | | | | | | | |
| Zinc 4.82%) + | | | | | | | | | |
| Ferric | | | | | | | | | |
| Oxide % | | | | | | | | | |
| (elemental | | | | | | | | | |
| Iron 4.196%) | | | | | | | | | |
| Magnesium | | | | | | | | | |
| Oxide 12% | | | | | | | | | |
| (elemental | | | | | | | | | |
| Magnesium | | | | | | | | | |
| 7.23%)-SC % | | | | | | | | | |
| @ 500 g/ha | | | | | | | | | |
| T5-Zinc | | 24.10 | 0.00 | 0.00 | 1.90 | 1.20 | 55.00 | 2105 | 10.79 |
| Oxide 6% | | | | | | | | | |
| (elemental | | | | | | | | | |
| Zinc 4.82%) | | | | | | | | | |
| WDG @ | | | | | | | | | |
| 500 g/ha | | | | | | | | | |
| T6-Ferric | | 0.00 | 20.98 | 0.00 | 2.50 | 2.10 | 50.00 | 2200 | 15.79 |
| Oxide 6% | | | | | | | | | |
| (elemental | | | | | | | | | |
| Iron 4.196%) | | | | | | | | | |
| WDG @ | | | | | | | | | |
| 500 g/ha | | | | | | | | | |
| T7- | | 0.00 | 0.00 | 36.15 | 1.60 | 1.10 | 70.00 | 2300 | 21.05 |
| Magnesium | | | | | | | | | |
| Oxide 12% | | | | | | | | | |
| (elemental | | | | | | | | | |
| Magnesium | | | | | | | | | |
| 7.23%) | | | | | | | | | |
| WDG @ | | | | | | | | | |
| 500 g/ha | | | | | | | | | |
| T8- | | | | | 0.8 | 0.9 | 50 | 1900 | |
| Untreated | | | | | | | | | |

It can be seen from the data presented in Table 3 that Treatment T1 (liquid suspension composition of Zinc Oxide 6%+Ferric Oxide 6%+Magnesium Oxide 12% with particle size in the range of 0.1 micron to 20 microns as per the embodiment of the present invention showed a significant increase in the yield when compared to treatment T2 with Zinc Oxide 6%+Ferric Oxide 6%+Magnesium Oxide 12% liquid suspension having particle size in the range of 0.1 to 50 microns, T3 with Zinc Oxide 6%+Ferric Oxide 6%+Magnesium Oxide 12% liquid suspension having particle size in the range of 21 to 50 microns and T4 with Zinc Oxide 6%+Ferric Oxide 6%+Magnesium Oxide 12% liquid suspension having particle size in the range of 50 to 100 microns. It was observed that the Treatment T1 showed a surprisingly significant 84.21% increase in the yield whereas the treatments T2, T3 and T4 only showed a yield increase of 40.53%, 36.84% and 35.26%, respectively as compared to the untreated control.

Further, the uptake of nutrients such as Iron, Magnesium, Zinc was found to be very high with the Treatment T1 as compared to Treatments T2, T3 and T4. It was thus noted that the superior efficacy in terms of yield and uptake of nutrients was observed with the liquid suspension formulation as per the present invention, where the composition comprised particles in the size range of 0.1 micron-20 microns when compared to liquid suspension formulations with higher particle size ranges.

### Experiment 4: To Assess the Efficacy of Different Formulations of “Water-Insoluble Iron Salt, Water-Insoluble Zinc Salt and Water-Insoluble Magnesium Salt” in Commercial Cultivated Wheat Crop Field:

### Field Experiment Methodology:

The trial was laid out during Rabi season in Randomized Block Design (RBD) with thirteen treatments including untreated control, replicated four times. For each treatment, plot size of 30 sq·m (6 m×5 m) was maintained. The compositions evaluated include Zinc salt, Iron salt, Magnesium salt alone and different formulations including combinations of water insoluble Zinc salt, water insoluble Iron salt, water insoluble Magnesium salt, where Zinc salt, Iron salt, Magnesium salt were applied in each treatment at same dosages. The Wheat crop in the trial field was raised following good agricultural practices. The details of the experiment are as follows:

Details of experiment

| a) Trial Location | Karnal, Haryana |
|---|---|
| b) Crop & Variety | Wheat (var: PBW 343) |
| c) Experiment season | Rabi 2022 |
| d) Trial Design | Randomized Block Design |
| e) Replications | 4 |
| f) Treatment | 13 |
| g) Plot size | 6 m × 5 m = 30 sq. m |
| h) Date of sowing | 9 Nov. 2022 |
| i) Date of Application | 25 Nov. 2022 |
| j) Method of application | Soil application |
| k) Date of Harvesting | 11 Apr. 2022 |
| l) Soil pH | 7-7.5 |

The mean data of all the observations were presented in Table 4 to illustrate the impact of combination comprising water insoluble salt of Zinc, water insoluble salt of Magnesium and water insoluble salt of Iron in liquid suspension form as per the embodiment of the present invention as well as in pastille form and powder form, on Wheat yield and other parameters.

TABLE 4

| | Dosage of Active | | | Wheat | % | Expected | Plant | Number |
|---|---|---|---|---|---|---|---|---|
| | content (g/hectare) | | | Yield | Increase | % Increase | Height at | of |
| Treatment Details | Zinc | Iron | Magnesium | (Qt/ha) | in Yield | in Yield | 30 DAA | Tillers |
| T1- Zinc Silicate | 46.93 | 19.28 | 13.95 | 19 | 26.67 | | 22 | 4 |
| 20% (elemental Zinc | | | | | | | | |
| 11.73%) + Ferrous | | | | | | | | |
| Carbonate 10% | | | | | | | | |
| (elemental Iron | | | | | | | | |
| 4.82%) + | | | | | | | | |
| Magnesium Silicate | | | | | | | | |
| Hydrate 20% | | | | | | | | |
| (elemental | | | | | | | | |
| Magnesium | | | | | | | | |
| 3.486%)-Pellet | | | | | | | | |
| @400 g/ha | | | | | | | | |
| T2- Zinc Silicate | 46.93 | 19.28 | 13.95 | 20 | 33.33 | | 23 | 3 |
| 20% (elemental Zinc | | | | | | | | |
| 11.73%) + Ferrous | | | | | | | | |
| Carbonate 10% | | | | | | | | |
| (elemental Iron | | | | | | | | |
| 4.82%) + | | | | | | | | |
| Magnesium Silicate | | | | | | | | |
| Hydrate 20% | | | | | | | | |
| (elemental | | | | | | | | |
| Magnesium | | | | | | | | |
| 3.486%)-Powder | | | | | | | | |
| @400 g/ha | | | | | | | | |
| T3- Zinc Silicate | 46.93 | 19.28 | 13.95 | 24.5 | 63.33 | 36.89 | 32 | 7 |
| 20% (elemental Zinc | | | | | | | | |
| 11.73%) + Ferrous | | | | | | | | |
| Carbonate 10% | | | | | | | | |
| (elemental Iron | | | | | | | | |
| 4.82%) + | | | | | | | | |
| Magnesium Silicate | | | | | | | | |
| Hydrate 20% | | | | | | | | |
| (elemental | | | | | | | | |
| Magnesium | | | | | | | | |
| 3.486%)-SC | | | | | | | | |
| @400 g/ha | | | | | | | | |
| T4-Zinc Silicate | 46.93 | 0.00 | 0.00 | 17 | 13.33 | | 19.50 | 3.00 |
| 20% (elemental Zinc | | | | | | | | |
| 11.73%) SC | | | | | | | | |
| @400 g/ha | | | | | | | | |
| T5- Ferrous | 0.00 | 19.97 | 0.00 | 18 | 20.00 | | 19.00 | 4.00 |
| Carbonate 10% | | | | | | | | |
| (elemental Iron | | | | | | | | |
| 4.82%) SC | | | | | | | | |
| @400 g/ha | | | | | | | | |
| T6- Magnesium | 0.00 | 0.00 | 13.95 | 16 | 6.67 | | 21.00 | 3.50 |
| Silicate Hydrate- | | | | | | | | |
| 20% (elemental | | | | | | | | |
| Magnesiun 3.486%) | | | | | | | | |
| SC @400 g/ha | | | | | | | | |
| T7- Zinc Oxide 10% | 64.27 | 31.10 | 34.87 | 20 | 33.33 | | 27 | 3.8 |
| (elemental Zinc | | | | | | | | |
| 8.033%) + Iron (II) | | | | | | | | |
| Oxide 5% | | | | | | | | |
| (elemental Iron | | | | | | | | |
| 3.886%) + | | | | | | | | |
| Magnesium Silicate | | | | | | | | |
| Hydrate 25% | | | | | | | | |
| (elemental | | | | | | | | |
| Magnesium 4.35%) - | | | | | | | | |
| Pellet @800 g/ha | | | | | | | | |
| T8- Zinc Oxide 10% | 64.27 | 31.10 | 34.87 | 22 | 46.67 | | 25 | 5 |
| (elemental Zinc | | | | | | | | |
| 8.033%) + Iron (II) | | | | | | | | |
| Oxide 5% | | | | | | | | |
| (elemental Iron | | | | | | | | |
| 3.886%) + | | | | | | | | |
| Magnesium Silicate | | | | | | | | |
| Hydrate 25% | | | | | | | | |
| (elemental | | | | | | | | |
| Magnesium 4.35%) - | | | | | | | | |
| Powder @800 g/ha | | | | | | | | |
| T9- Zinc Oxide 10% | 64.27 | 31.10 | 34.87 | 28 | 86.67 | 58.67 | 45 | 8 |
| (elemental Zinc | | | | | | | | |
| 8.033%) + Iron (II) | | | | | | | | |
| Oxide 5% | | | | | | | | |
| (elemental Iron | | | | | | | | |
| 3.886%) + | | | | | | | | |
| Magnesium Silicate | | | | | | | | |
| Hydrate 25% | | | | | | | | |
| (elemental | | | | | | | | |
| Magnesium 4.35%) - | | | | | | | | |
| SC@800 g/ha | | | | | | | | |
| T10-Zinc Oxide | 64.27 | 0.00 | 0.00 | 18 | 20.00 | | 19.00 | 3.50 |
| 10% (elemental Zinc | | | | | | | | |
| 8.033%) SC | | | | | | | | |
| @800 g/ha | | | | | | | | |
| T11- Iron (II) Oxide | 0.00 | 31.10 | 0.00 | 17 | 13.33 | | 21.00 | 3.00 |
| 5% (elemental Iron | | | | | | | | |
| 3.886%) SC | | | | | | | | |
| @800 g/ha | | | | | | | | |
| T12- Magnesium | | 0.00 | 34.87 | 19.5 | 30.00 | | 22.00 | 4.00 |
| Silicate Hydrate | | | | | | | | |
| 25% (elemental | | | | | | | | |
| Magnesium 4.35%) | | | | | | | | |
| SC @800 g/ha | | | | | | | | |
| T13-Untreated | | | | 15 | | | 18.00 | 2.50 |

It can be clearly seen from the Table 4 above that the treatment T3 with Zinc Silicate 20% (elemental Zinc 11.73)+Ferrous Carbonate 10% (elemental Iron 4.82%)+Magnesium Silicate Hydrate 20% (elemental Magnesium 3.486%)—SC, as per the embodiment of the present invention showed a yield increase of 63.33% in Wheat grain yield. However, treatment T1 with Zinc Silicate 20%+Ferrous Carbonate 10%+Magnesium Silicate Hydrate 20%-Pellet demonstrated only an increase of 26.67% while treatment T2 with Zinc Silicate 20%+Ferrous Carbonate 10%+Magnesium Silicate Hydrate 20% Powder demonstrated only an increase of 33.33% in the grain yield. Based on the data and the calculations made by referring the treatments T1-T6, the expected percentage increase in the fruit yield was 36.89%. Thus, it can be noted that the treatment T3—SC as per the present invention demonstrated a synergistic effect, as compared to the same treatment with pastille or with powder compositions. i.e. Treatments T1, T2 respectively as well as the application of individual actives i.e. Treatments T4-T6 despite being applied at same dosage of applications of Zinc, Iron and Magnesium respectively. The results are all the more surprising as all the treatments T1 to T6 had the same dosage of Zinc, Iron and Magnesium being applied to the soil i.e. 46.93 gm/ha of Zinc, 19.28 gm/ha of Iron and 13.95 gm/ha of Magnesium.

Further, treatment T9 with Zinc Oxide 10% (elemental Zinc 8.033%)+Iron (II) Oxide 5% (elemental Iron 3.886%)+Magnesium Silicate Hydrate 25% (elemental Magnesium 4.35%)—SC exhibited highest grain yield of about 86.67% when compared to treatment T7 with Zinc Oxide 10%+Iron (II) Oxide 5%+Magnesium Silicate Hydrat-Pellet (a grain yield of 33.33%), Treatment T8 with Zinc Oxide 10%+Iron (II) Oxide 5%+Magnesium Silicate Hydratate 25%-Powder (a grain yield of 46.67%). It was further observed that treatments T3 and T9 with composition as per the embodiment of the present invention showed increased greenness and improved plant height and number of tillers, as compared to pastille and powder compositions i.e. treatments T1-T2 and T7-T8 respectively.

It was thus noted that composition of “water insoluble Iron salt, water insoluble Zinc salt and water insoluble Magnesium salt” in the form of a suspension concentrate as per the embodiments of the present invention is synergistic in nature and showed a surprising enhancement in the yield as well as improved plant physiological parameters as compared to other known formulation types.

### Experiment No. 5: To Study the Effect of Suspension Concentrate Composition of Present Invention in Maize:

The trial was laid out during Kharif season in Randomized Block Design (RBD) with six treatments including untreated control, replicated four times. The compositions evaluated include SC composition of Zinc salt and Iron salt and SC composition of the present invention as soil application after planting of Maize seedlings in the trial plot. The Maize crop in the trial field was raised following good agricultural practices.

Details of experiment

| a) Trial Location | Davangere, Karnataka |
|---|---|
| b) Crop & Variety | NK 7720 (Syngenta) |
| c) Experiment season | Kharif 2022 |
| d) Trial Design | Randomized Block Design |
| e) Replications | 4 |
| f) Treatment | 6 |
| g) Plot size | 6 m × 5 m = 30 sq. m |
| h) Date of sowing | 12 Jul. 2022 |
| i) Date of Application | 12 Jul. 2022 |
| j) Method of application | Soil application |
| k) Date of Harvesting | 20 Nov. 2022 |
| l) Soil pH | 6.5-7 |

The observations were recorded at the harvesting time and the mean data was presented in Table 5 to enumerate the efficacy of the suspension concentrate comprising “Water insoluble Zinc salt and Water insoluble Magnesium salt and water insoluble Iron salt” prepared as per the embodiment of the present invention.

TABLE 5

| | Formulation | Dosage of Active | | | | % | Nutrient | | |
|---|---|---|---|---|---|---|---|---|---|
| Treatment | dosage | ingredients (g/ha) | | | Yield | Increase | concentration(mg/100 gm) | | |
| Details | in g/ha | Zinc | Iron | Magnesium | (Kg/ha) | in Yield | Zn | Fe | Mg |
| T1- Zinc Borate | 1000.0 | 156.55 | 104.98 | | 3279 | 11.15 | 1.1 | 1.0 | 6.7 |
| 25% (elemental | | | | | | | | | |
| Zinc 15.62%) + | | | | | | | | | |
| Ferric Oxide 15% | | | | | | | | | |
| (elemental Iron | | | | | | | | | |
| 10.49%) SC | | | | | | | | | |
| T2- Zinc Borate | 1000.0 | 156.55 | 104.98 | 43.58 | 4438 | 50.44 | 5.3 | 4.3 | 21.3 |
| 25% (elemental | | | | | | | | | |
| Zinc 15.62%) + | | | | | | | | | |
| Ferric Oxide 15% | | | | | | | | | |
| (elemental Iron | | | | | | | | | |
| 10.49%) + | | | | | | | | | |
| Magnesium | | | | | | | | | |
| Silicate 25% | | | | | | | | | |
| (elemental | | | | | | | | | |
| Magnesium | | | | | | | | | |
| 4.358%) -SC | | | | | | | | | |
| T3- Zinc Sulfide | 1000.0 | 53.66 | 15.55 | 0.00 | 3410 | 15.59 | 0.9 | 0.8 | 7.5 |
| 8% (elemental | | | | | | | | | |
| Zinc 5.366%) + | | | | | | | | | |
| Iron (II) Oxide | | | | | | | | | |
| 2% (elemental | | | | | | | | | |
| Iron 1.55%) SC | | | | | | | | | |
| T4- Zinc Sulfide | 1000.0 | 53.66 | 15.55 | 66.68 | 4500 | 52.54 | 5.1 | 4.1 | 20.3 |
| 8% (elemental | | | | | | | | | |
| Zinc 5.366%) + | | | | | | | | | |
| Iron (II) Oxide | | | | | | | | | |
| 2% (elemental | | | | | | | | | |
| Iron 1.55%) + | | | | | | | | | |
| Magnesium | | | | | | | | | |
| Hydroxide 16% | | | | | | | | | |
| (elemental | | | | | | | | | |
| Magnesium | | | | | | | | | |
| 6.668%) -SC | | | | | | | | | |
| T5-Commercial | 30000 | | | | 3510 | 18.98 | 1.2 | 0.8 | 10.6 |
| multi-nutrient | | | | | | | | | |
| sample | | | | | | | | | |
| (Microfood T - | | | | | | | | | |
| Stanes (basal))- | | | | | | | | | |
| Powder | | | | | | | | | |
| T6-Untreated | | | | | 2950 | | 1.3 | 0.3 | 9.8 |

It can be observed from Table 5 that the SC composition comprising Zinc salt, Iron salt and Magnesium salt as per the embodiment of the present invention shows a significant enhancement in the uptake of nutrients as compared to the uptake observed with the application of SC composition of Zinc salt+Iron salt at an acidic soil pH condition where the active Zn and Fe were applied at same active dosage. For instance: Treatment T2 with Zinc Borate 25% (elemental Zinc 15.626%)+Ferric Oxide 15% (elemental Iron 10.49%)+Magnesium Silicate 25% (elemental Magnesium 4.358%)—SC prepared as per the embodiment of the present invention demonstrates an uptake of 5.3 mg of Zinc, 4.3 mg of Iron and 21.3 mg of Magnesium while treatment T1 with—Zinc Borate 25%+Ferric Oxide 15% SC which is devoid of Magnesium shows a reduced uptake of 1.1 mg of Zinc, 1.0 mg of Iron and 6.7 mg of Magnesium where the uptake of zinc and iron was found to be very low even at acidic soil pH which is generally considered favorable for nutrient uptake. This appreciable increase in the availability of Zinc and Iron observed in Treatment T2 was noted to be on account of the presence of Magnesium along with Zinc and Iron in the composition formulated as per the embodiment of the invention i.e., in the form of a liquid suspension with particle size in the range of 0.1 microns to 20 microns in Treatment T2 which facilitated the increased availability of the entire range of micronutrients present in the composition i.e. Magnesium Iron and Zinc for uptake by the crops.

Further, the application of Treatment 4 with Zinc Sulfide 8% (elemental Zinc 5.366%)+Iron (II) Oxide 2% (elemental Iron 1.55%)+Magnesium Hydroxide 16% (elemental Magnesium 6.668%)—SC according to an embodiment of the present invention showed an increase of 52.54% in yield of Maize as compared to treatment T3 which showed 15.59% in Maize grain yield. The enhancement in efficacy with the composition as per the embodiment of the present invention is surprising as the dosages of Zinc and Iron applied in SC composition as per the present invention as well as the two-way treatment which is devoid of Magnesium i.e. T3 are the same.

The surprising efficacy observed in terms of grain yield of Maize for the treatments T2 and T4 where the three actives are present as per the embodiment of the present invention i.e., in a single composition and in specific concentration, wherein the composition comprises particles in the size range of 0.1 micron to 20 microns. It was further noted that the treatments T2 and T4 with a yield increase of about 50.44% and 52.54% respectively demonstrated higher efficacy when compared to the commercially available micronutrient mixture i.e. Treatment T5 which despite being applied at high formulation dosage showed a yield increase of only 18.98%.

It was further observed that with Treatment T5, the uptake of Zinc, Iron and Magnesium was found to be 1.2 mg, 0.8 mg and 10.6 mg respectively and with Treatment T3, the uptake of Zinc, Iron and Magnesium was found to be 0.9 mg, 0.8 mg, and 7.5 mg respectively. On the other hand, with Treatment T4 the uptake of Zinc, Iron and Magnesium was found to be 5.1 mg, 4.1 mg and 20.3 mg respectively. This appreciable increase in the availability of Zinc and Iron observed in Treatment T4 was noted to be on account of the presence of Magnesium along with Zinc and Iron in the composition formulated as per the embodiment of the invention i.e., in the form of a liquid suspension with particle size in the range of 0.1 microns to 20 microns facilitated the increased availability of the entire range of micronutrients present in the composition i.e., Magnesium Iron and Zinc for uptake by the crops. It was thus noted that the composition comprising a combination of water insoluble Zinc salt and water insoluble Magnesium salt and water insoluble Iron salt in the form of water dispersible granules demonstrates a better uptake of Magnesium, Zinc and Iron when compared to commercially available multi-nutrient powder composition as well as an application of a composition of only Iron and Zinc which is devoid of Magnesium.

From the aforementioned data, it can be concluded that the composition comprising of “water insoluble Zinc salt and water insoluble Magnesium salt and water insoluble Iron salt” in the form of WDG as per the embodiment of the present invention at different dosages and at claimed concentration ranges demonstrated significantly higher uptake of micronutrients, higher yield.

Thus, a composition of present invention in the form of a liquid suspension composition was found to be high nutrient use efficient fertilizer.

### Experiment No 6—to Study Efficacy of Various Composition of Water Insoluble Salts of Zinc, Water Insoluble Salt of Iron and Water Insoluble Salt of Magnesium on Tomato Crop.

The field trials were carried out to observe the effect of various formulations of water insoluble salts of Zinc, water insoluble salt of Iron and water insoluble salt of Magnesium on yield and yield attributing parameters in Tomato at Niphad, Maharashtra. The trial was laid out during Kharif season in Randomized Block Design (RBD) comprising seven treatments as described below including untreated control, replicated three times.

For each treatment, plot size of 40 sq·m (8 m×5 m) was maintained. The treatments as per prescribed dose were applied as foliar at 30 days after planting of the tomato crops and spray application was repeated twice a week. The tomato crop in trial field was raised following good agricultural practices.

Details of experiment

| | |
|---|---|
| a) Trial Location | Niphad (Maharashtra) |
| b) Crop | Tomato (Avinash) |
| c) Experiment season | Kharif 2022 (June to October) |
| d) Trial Design | Randomized Block Design |
| e) Replications | Three |
| f) Treatment | Ten |
| g) Plot size | 8 m × 5 m = 40 sq. m |
| h) Date of sowing | 4 Jun. 2022 |
| i) Date of Application | 1st-5 Jul. 2022; 2nd-14 Jul. 2022; 3rd-25 |
| | Jul. 2022 |
| j) Method of application | Foliar spray using water volume @ 500 L/ha |

The observations were recorded and mean data is presented in tables 6 to enumerate the impact of different treatments.

TABLE 6

| | | | | | | | Expected | Mean | |
|---|---|---|---|---|---|---|---|---|---|
| | | | | | Fruit | % | % | No. | |
| | Formulation | Dosage of nutrients | | | yield | Increase | Increase | of | Fruit |
| Treatment | dosage | gm/ha | | | (Qtl/ | in | in | flower/ | weight |
| Details | in g/ha | Zinc | Iron | Magnesium | acre) | Yield | Yield | bunch* | (Kg) |
| T1- Zinc | 250.0 | 13.03 | | | 85 | 13.33 | 35.70* | 4.1 | 8 |
| Carbonate | | | | | | | | | |
| (elemental | | | | | | | | | |
| Zinc | | | | | | | | | |
| 5.2%)10-% | | | | | | | | | |
| OD | | | | | | | | | |
| T2- Ferrous | 250.0 | 0.00 | 6.03 | | 80 | 6.67 | | 3.9 | 6.5 |
| Carbonate | | | | | | | | | |
| 5% | | | | | | | | | |
| (elemental | | | | | | | | | |
| Iron 2.41%) | | | | | | | | | |
| OD | | | | | | | | | |
| T3- | 250.0 | 0.00 | 0.00 | 22.62 | 89 | 18.67 | | 4.5 | 7 |
| Magnesium | | | | | | | | | |
| Oxide 15% | | | | | | | | | |
| (elemental | | | | | | | | | |
| Magnesium | | | | | | | | | |
| 9.04%) OD | | | | | | | | | |
| T4-Zinc | 250.0 | 13.03 | 6.03 | 22.62 | 147 | 96.00 | | 5.9 | 9.16 |
| Carbonate | | | | | | | | | |
| 10% | | | | | | | | | |
| (elemental | | | | | | | | | |
| Zinc 5.2%) + | | | | | | | | | |
| Ferrous | | | | | | | | | |
| Carbonate | | | | | | | | | |
| 5% | | | | | | | | | |
| (elemental | | | | | | | | | |
| Iron 2.41%) + | | | | | | | | | |
| Magnesium | | | | | | | | | |
| Oxide 15% | | | | | | | | | |
| (elemental | | | | | | | | | |
| Magnesium | | | | | | | | | |
| 9.04%) OD | | | | | | | | | |
| T5- Zinc | 500.0 | 3.81 | | | 79 | 5.33 | 26.21* | 3.9 | 7 |
| Phosphate | | | | | | | | | |
| 2.5% | | | | | | | | | |
| (elemental | | | | | | | | | |
| Zinc 1.277%) | | | | | | | | | |
| SE | | | | | | | | | |
| T6- Iron | 300.0 | | 4.93 | | 82 | 9.33 | | 4.1 | 7.5 |
| Silicate 5% | | | | | | | | | |
| (elemental | | | | | | | | | |
| Iron 1.642%) | | | | | | | | | |
| SE | | | | | | | | | |
| T7- | 300.0 | 0.00 | | 15.63 | 85 | 13.33 | | 4.5 | 7.9 |
| Magnesium | | | | | | | | | |
| Hydroxide | | | | | | | | | |
| 12.5% | | | | | | | | | |
| (elemental | | | | | | | | | |
| Magneisum | | | | | | | | | |
| 5.20%) SE | | | | | | | | | |
| T8-Zinc | 300 | 3.81 | 4.93 | 15.63 | 135 | 80.00 | | 5.1 | 8.5 |
| Phosphate | | | | | | | | | |
| 2.5-% | | | | | | | | | |
| (elemental | | | | | | | | | |
| Zinc 1.277%) + | | | | | | | | | |
| Iron | | | | | | | | | |
| Silicate 5% + | | | | | | | | | |
| Magnesium | | | | | | | | | |
| Hydroxide | | | | | | | | | |
| 12.5% | | | | | | | | | |
| (elemental | | | | | | | | | |
| Magnesium | | | | | | | | | |
| 5.20%) SE | | | | | | | | | |
| T9-Untreated | | | | | 75 | | | 3.5 | 6 |

SE* Suspoemulsion
OD* Oil Dispersion
*Mean of 5 bunch/plant and 10 plants each plot

From Table 6, it was observed that the composition comprising water insoluble salts of Zinc, water insoluble salt of Iron and water insoluble salt of Magnesium in form of oil dispersion and suspoemulsion is synergistic in nature.

It can be observed from Table 6 that treatment T4-Zinc Carbonate 10% (elemental Zinc 5.2%)+Ferrous Carbonate 5% (elemental Iron 2.41%)+Magnesium Oxide 15% (elemental Magnesium 9.04%) OD and treatment T8-Zinc Phosphate 2.5-%+Iron Silicate 5%+Magnesium Hydroxide 12.5% SE composition prepared according to an embodiment of the present invention demonstrated better yield in as compared to treatments T1-13 and T5-T7 respectively as well as over the untreated plot.

Thus, from the data presented in Table 6, it can be noted that the OD and SE compositions prepared as per embodiment of the present invention are synergistic and provides higher crop yield as compared to the application of individual actives when applied at the same dosage.

### Experiment No. 7: To Study the Effect of the Composition of the Present Invention on Uptake of Zinc, Magnesium and Iron in Different Soil pH Conditions.

Pot trial experiments were carried out to observe the effect of the composition of the present invention in the form of WDG on the availability of Zinc, Magnesium and Iron in different soil types over a period of time on Onion Crop in Poly-house at Junagadh (Gujarat) (India).

The 5 pots, sized with 20 cm top diameter×15.5 cm bottom diameter×16.5 cm height, for each treatment were arranged in Randomized Block Design (RBD) and labelled in order to make three treatment for each experiment.

The Test Nutritional compositions as indicated below at prescribed dose were measured based on surface area calculation of soil and applied in the respective treatment pots on top soil and mixed in soil well up to 5 cm depth. Thereafter, a 25 days old onion seedling was planted in each pot. The planted onion seedlings in the 5 pots were raised with GAP (Good Agricultural Practice) until harvesting or full development of Onion bulb.

The Treatment Details are as follows:
T1—Zinc Oxide 8%+Iron (III) Oxide 4%+Magnesium Silicate Hydrate 20%—SC
T2—Zinc Oxide 8%+Iron (III) Oxide 4%—SC

The Details of the Experiment are as follows:

| | |
|---|---|
| a) Trial Location | Junagardh Gujarat (Maharashtra |
| b) Crop | Onion |
| Experiment season | Rabi 2021-2022 |
| d) Trial Design | Randomized Block Design with 5 pot in each |
| | treatment |
| e) Replications | 13 |
| f) Treatment | 7 |
| g) Pot size | 20 cm top diameter × 15.5 cm bottom |
| | diameter × 16.5 cm height |
| h) Date of Application | 22 Nov. 2021 |
| i) Date of seedling planting | 22 Nov. 2021 |
| j) Method of application | Basal (Soil Application) |
| k) Date of Harvesting | 2 Mar. 2022 |

The observations on uptake on nutrients were recorded at the harvesting time and mean data was presented in Tables 7A, 7B, 7C to enumerate the availability of Zinc, Magnesium and Iron in different pH conditions.

TABLE 7A
Tests were performed in Alkaline Soil (pH-8.5 to 9) to assess the nutrient availability
from different treatments and mean values were presented as below

| | | Active ingredients (g/ha) | | | Nutrient concentration | | |
|---|---|---|---|---|---|---|---|
| | Formulation | Zinc | Iron | Magnesium | in mg/100 g of Onion | | |
| | dosage in | (Zn) | (Fe) | (Mg) | Bulb | | |
| Compositions | g/ha | content | content | content | Mg | Fe | Zn |
| T1- Zinc | 1500 | 96.41 | 41.9 | 52.20 | 10.5 | 1.2 | 1 |
| Oxide 8% | | | | | | | |
| (elemental | | | | | | | |
| Zinc 6.427%) + | | | | | | | |
| Iron (III) | | | | | | | |
| Oxide 4% | | | | | | | |
| (elemental | | | | | | | |
| Iron 2.79%) + | | | | | | | |
| Magnesium | | | | | | | |
| Silicate | | | | | | | |
| Hydrate 20% | | | | | | | |
| (elemental | | | | | | | |
| Magnesium | | | | | | | |
| 3.48%) -SC | | | | | | | |
| prepared as | | | | | | | |
| per the | | | | | | | |
| embodiment | | | | | | | |
| of the present | | | | | | | |
| invention | | | | | | | |
| T2- Zinc | 1500 | 96.41 | 41.9 | — | 1.5 | 0.06 | 0.08 |
| Oxide 8% | | | | | | | |
| (elemental | | | | | | | |
| Zinc 6.427%) + | | | | | | | |
| Iron (III) | | | | | | | |
| Oxide 4% | | | | | | | |
| (elemental | | | | | | | |
| Iron 2.79%) | | | | | | | |
| SC | | | | | | | |
| T3-Untreated | — | — | — | — | 0.3 | 0.01 | 0.02 |

TABLE 7B
Tests were performed in Acidic soil (pH-6 to 6.5) to assess the nutrient availability
from different treatments and mean values were presented as below

| | | Active ingredients (g/ha) | | | Nutrient concentration | | |
|---|---|---|---|---|---|---|---|
| | Formulation | Zinc | Iron | Magnesium | in mg/100 g of Onion | | |
| | dosage in | (Zn) | (Fe) | (Mg) | Bulb | | |
| Compositions | g/ha | content | content | content | Mg | Fe | Zn |
| T1- Zinc | 1500 | 96.41 | 41.96 | 52.20 | 13 | 1.8 | 1.4 |
| Oxide 8% | | | | | | | |
| (elemental | | | | | | | |
| Zinc 6.427%) + | | | | | | | |
| Iron (III) | | | | | | | |
| Oxide 4% | | | | | | | |
| (elemental | | | | | | | |
| Iron 2.79%) + | | | | | | | |
| Magnesium | | | | | | | |
| Silicate | | | | | | | |
| Hydrate 20% | | | | | | | |
| (elemental | | | | | | | |
| Magnesium | | | | | | | |
| 3.48%) SC | | | | | | | |
| prepared as | | | | | | | |
| per the | | | | | | | |
| embodiment | | | | | | | |
| of the present | | | | | | | |
| invention | | | | | | | |
| T2- Zinc | 1500 | 96.41 | 41.96 | | 2.0 | 0.2 | 0.3 |
| Oxide 8% | | | | | | | |
| (elemental | | | | | | | |
| Zinc 6.427%) + | | | | | | | |
| Iron (III) | | | | | | | |
| Oxide 4% | | | | | | | |
| (elemental | | | | | | | |
| Iron 2.79%) | | | | | | | |
| SC | | | | | | | |
| T3-Untreated | — | — | — | — | 2.0 | 0.04 | 0.06 |

TABLE 7C
Tests were performed in Neutral Soil (pH-7) to assess the nutrient availability
from different treatments and mean values were presented as below

| | | Active ingredients (g/ha) | | | Nutrient concentration | | |
|---|---|---|---|---|---|---|---|
| | Formulation | Zinc | Iron | Magnesium | in mg/100 g of Onion | | |
| | dosage in | (Zn) | (Fe) | (Mg) | Bulb | | |
| Compositions | g/ha | content | content | content | Mg | Fe | Zn |
| T1- Zinc Oxide 8% | 1500 | 96.41 | 41.96 | 52.20 | 17 | 2.9 | 1.7 |
| (elemental Zinc | | | | | | | |
| 6.427%) + Iron (III) | | | | | | | |
| Oxide 4% (elemental | | | | | | | |
| Iron 2.79%) + | | | | | | | |
| Magnesium Silicate | | | | | | | |
| Hydrate 20% (elemental | | | | | | | |
| Magnesium 3.48%) SC | | | | | | | |
| prepared as per the | | | | | | | |
| embodiment of the | | | | | | | |
| present invention | | | | | | | |
| T2- Zinc Oxide 8% | 1500 | 96.41 | 41.96 | — | 1.9 | 0.2 | 0.3 |
| (elemental Zinc | | | | | | | |
| 6.427%) + Iron (III) | | | | | | | |
| Oxide 4% (elemental | | | | | | | |
| Iron 2.79%) SC | | | | | | | |
| T3-Untreated | — | — | — | — | 0.09 | 0.02 | 0.05 |

From Table 7B & Table 7C, it was noted that Zinc and Iron were moderately available for uptake with Treatment T2 when applied in both acidic soil and neutral soil pH respectively as compared to those observed with Treatment T2 (Table 7A) where the soil pH was alkaline despite the same treatment i.e. Zinc Oxide 8%+Iron (III) Oxide 4%—SC was applied at same active dosage of Zinc and Iron.

It was further observed from Tables 7A, 7B and 7C that when treatment T1 with Zinc Oxide 8% (elemental Zinc 6.427%)+Iron (III) Oxide 4% (elemental Iron 2.79%)+Magnesium Silicate Hydrate 20% (elemental Magnesium 3.48%) SC as per the embodiment of the present invention was applied, the uptake of nutrients like Zinc, Magnesium and Iron was found to be comparatively same at all soil pH conditions. Upon comparing the results presented for the Treatments T1, T2 of Table 7A, it was further surprising to observe that the uptake of Zinc and Iron was found to be substantially increased with Treatment T1 (as per the embodiment of the present invention) where Magnesium Silicate Hydrate was added to the composition of Zinc Oxide 8%+Iron (III) Oxide 4%—SC despite the pH being alkaline, which was not observed with treatment T2.

It is thus noted that, the liquid suspension composition of “water insoluble Iron salt, water insoluble zinc salt and water insoluble Magnesium salt” as per the embodiments of the present invention depicts significantly higher uptake of Iron and Zinc even at alkaline pH which was not observed with two-way mixture of Iron salt and Zinc salt at same pH. It can be appreciated from the observed results that on account of the presence of Magnesium along with Zinc and Iron in the composition formulated as per the embodiment of the invention i.e., in the form of a liquid suspension with particle size in the range of 0.1 microns to 20 microns facilitates the uptake of Iron and Zinc in alkaline soil which was not observed with the composition devoid of Magnesium i.e. treatment T2.

Further, the Treatment T1-Zinc Oxide 8% (elemental Zinc 6.427%)+Iron (III) Oxide 4% (elemental Iron 2.79%)+Magnesium Silicate Hydrate 20% (elemental Magnesium 3.48%) SC as per the embodiments of the present invention was found to be nutrient use efficient and demonstrates good update of all the three nutrients in acidic, neutral as well as alkaline pH soil conditions.

Experiment No. 8: To Compare the Effect of Composition of Present Invention Vis-à-Vis Commercially Available Water-Soluble Powder of Multi-Nutrient in Maize Crop:

The field trial was carried out on a commercially cultivated Maize field at Nashik in Maharashtra to compare the effect of a SC composition comprising a combination of water insoluble salts of Zinc, Magnesium and Iron vis-a-vis commercially available water-soluble multi-nutrient powder, a product “SPIC Nourish” (comprising Zn, Fe, Mn, B, Mg, Cu) in Maize. The trial was laid out during Kharif season in Randomized Block Design (RBD) with three treatments including untreated control. The compositions of the present invention with prescribed dose were applied along with drip irrigation.

The Maize crop in trial field was raised following good agricultural practice

Details of experiment

| a) Trial Location | Nashik, Maharashtra |
|---|---|
| b) Crop & Variety | NK 7720 (Syngenta) |
| c) Experiment season | Kharif 2022 |
| d) Trial Design | Randomized Block Design |
| e) Replications | 4 |
| f) Treatment | 3 |
| g) Plot size | 6 m × 5 m = 30 sq. m |
| h) Date of sowing | 12 Jul. 2022 |
| i) Date of Application | 12 Jul. 2022 |
| j) Method of application | Soil application by drip irrigation |
| k) Date of Harvesting | 20 Nov. 2022 |
| l) Soil pH | 7-7.5 |

TABLE 8

| | Formulation | Maize Grain | |
|---|---|---|---|
| | Dose in | Yield | % Increase |
| Treatment Details | Kg/ha | (Kg/ha) | in Yield |
| T1- Zinc Phosphate 5% | 10 | 40100 | 45.8 |
| (elemental Zinc 2.55%) + | | | |
| Iron (II) Fumarate 5% | | | |
| (elemental Iron 1.64%) + | | | |
| Magnesium Hydroxide | | | |
| 15% (elemental | | | |
| Magnesium 6.25%) -SC as | | | |
| per the embodiment of | | | |
| present invention | | | |
| T2- SPIC Nourish - | 25 | 2980 | 8.36 |
| Commercially available | | | |
| water soluble multi- | | | |
| nutrient powder | | | |
| (comprising Zn, Fe, Mn, B, | | | |
| Mg, Cu) | | | |
| T3-Untreated | — | 2750 | — |

It can be observed from treatment T1 of Table 8 that a SC composition of Zinc Phosphate 5% (elemental Zinc 2.55%)+Iron (II) Fumarate 5% (elemental Iron 1.64%)+Magnesium Hydroxide 15% (elemental Magnesium 6.25%) prepared according to an embodiment of the present invention demonstrated better yield as compared to treatment T2 wherein the composition applied is a commercially available water soluble multi-nutrient mixture and the untreated plot. Treatment T1 depicted yield increase of about 45.8% despite being applied at reduced a dosage when compared to treatment T2 which had yield increase of only 8.36%. Thus, it can be concluded that even at a reduced dosage, the combination of “water insoluble Iron salt, water insoluble Zinc salt and water insoluble Magnesium salt” in the form of SC as per the embodiment of the present invention shows significant improvement in grain yield than that of the commercially available water soluble multi-nutrient mixture.

### Experiment 9: To Study the Effect of WDG Composition of the Present Invention in Comparison to Traditional Fertilizer Practices.

The field trials were carried out to determine the effect of composition of the present invention on availability of nutrients with that of the application of traditional fertilizer practices in calcareous soil at Junagadh, Gujarat (India) on Groundnut crop. The trials were laid down in Randomized Block Design (RBD) with three treatments including untreated control, replicated seven times. For each treatment, plot size of 40 sq·m (8 m×5 m) was maintained.

Soil was analyzed to assess nutrient availability before the date of application of treatment and the observations are as follow:

| N | P | K | S | Zn | Fe | Mn | Mg | Ca | B | Cu |
|---|---|---|---|---|---|---|---|---|---|---|
| 190 | 25 | 450 | 15.3 | 3.17 | 6.44 | 2.01 | 110 | 534 | 0.19 | 1.17 |
| kg/ha | kg/ha | kg/ha | ppm | ppm | ppm | ppm | ppm | ppm | ppm | ppm |

The Details of the Experiment are as follows:

| a) Trial Location | Rajkot (Gujarat) |
|---|---|
| b) Crop | GG20 |
| c) Experiment season | Kharif 2022 |
| d) Trial Design | Randomized Block Design with 5 pot in each |
| | treatment |
| e) Replications | 6 |
| f) Treatment | 4 |
| g) Pot size | 7 m × 5 m = 30 sq. m |
| h) Date of Application | 5 Jul. 2022 |
| i) Date of seedling planting | 5 Jul. 2022 |
| j) Method of application | Soil Application |
| k) Date of Harvesting | 10 Oct. 2022 |
| l)Soil pH | 7.5 |

The observations on the availability of nutrients in groundnut crop were recorded at the harvesting time and mean data were presented in Table 9 to enumerate the effect of composition of present invention in the calcareous soil.

TABLE 9

| | Formulation | Nutrient content in Groundnut | | | | | | NPK in leaves | | |
|---|---|---|---|---|---|---|---|---|---|---|
| | dosage in | (ppm) | | | | | | (Kg) | | |
| Treatment Details | g/ha | Zn | Fe | Mg | Mn | Ca | B | N | P | K |
| T1- Zinc Oxide | 500 | 2.2 | 6.1 | 130 | 1.9 | 300 | 0.17 | 105 | 20 | 180 |
| 10% (elemental | | | | | | | | | | |
| Zinc 8.033%) + | | | | | | | | | | |
| Ferric Oxide 15% | | | | | | | | | | |
| (elemental Iron | | | | | | | | | | |
| 10.492%) + | | | | | | | | | | |
| Magnesium | | | | | | | | | | |
| Silicate 30% | | | | | | | | | | |
| (elemental Iron | | | | | | | | | | |
| 5.23%) -SC | | | | | | | | | | |
| T2-NPK | 1000 | 0.9 | 1.2 | 45 | 1.1 | 150 | 0.03 | 80 | 11 | 105 |
| Traditional | | | | | | | | | | |
| fertilizer 19:19:19 | | | | | | | | | | |
| T3-Nutrifast (40% | 2500 | 0.1 | 1.7 | 35 | 1.4 | 105 | 0.05 | 75 | 9 | 110 |
| NPK + | | | | | | | | | | |
| 5% micronutrient) | | | | | | | | | | |
| T4-Untreated | | 0 | 0.1 | 20 | 0.4 | 100 | 0.01 | 70 | 8 | 90 |

It can be observed from Table 9 that treatment T1-Zinc Oxide 10% (elemental Zinc 8.033%)+Ferric Oxide 15% (elemental Iron 10.492%)+Magnesium Silicate 30% (elemental Iron 5.23%)—SC composition prepared according to an embodiment of the present invention demonstrated better uptake of nutrients in calcareous soil as compared to treatments T2 and T3 i.e. commercially available water soluble NPK fertilizers and commercially available NPK with water soluble micronutrient composition (Nutrifast by Stanes) respectively as well as over the untreated plot.

It was observed that the practice of application of NPK and NPK with other micronutrients such as Calcium, Boron, Manganese etc. even at higher dosage of application did not meet the nutritional requirement of the plant and failed to provide even an adequate uptake of Zinc, Iron and Magnesium along with other nutrients as observed with the composition of the present invention. Thus, it can be noted that despite being applied in calcareous soil, the combination of water insoluble Iron salt, water insoluble Zinc salt and water insoluble Magnesium salt as per the embodiment of the present invention shows significant nutrient availability to the plant as compared to treatments T2 and T3 i.e. synthetic fertilizer mixtures.

It can be appreciated from the observed results that on account of the presence of Magnesium along with Zinc and Iron in the composition formulated as per the embodiment of the invention i.e., in the form of a water dispersible granules with particle size in the range of 0.1 microns to 20 microns not only there is an uptake of Iron and Zinc in calcareous soil but also of other micronutrients including Manganese, Calcium, Boron etc, which was not observed with the commercially available water soluble NPK fertilizers and commercially available NPK with water soluble micronutrient composition i.e. treatments T2, T3. The present invention not only facilitates assimilation of essential nutrients like Magnesium, Zinc and Iron but also assist in unlocking the micronutrients and trace elements making them available for uptake by plants which were not available for uptake in mineral rich calcareous soil primarily because of reported antagonism between Ca—Mg, Ca—Fe and Ca—Zn.

Further, the inventors of the present invention also tested the SC, OD and SE composition of the present invention on other crops like Chili, Chickpea, Vegetables. It was observed that the composition of the present invention may further enhance crop characteristics like straw weight, plant height and also add to nutritional value of the crop. Further such combinations may additionally help in improving the crop yield, improved photosynthesis, increase chlorophyll content and uptake of nutrients by the crop.

It has been observed that the composition of the present invention, demonstrates enhanced, efficacious and superior behavior in the fields. Through the composition of the present invention, the number of applications or the amount of nutrients, fertilizers or pesticides are minimized. Moreover, the present composition exhibits a surprisingly higher field efficacy at reduced dosages of application of the composition as compared to prior known composition. The composition is highly safe for the user and for the environment. This novel composition helps to improve plant yield, balanced uptake of all nutrients, reduce yellowing of leaves and plant physiological parameters such as increased rooting, improved foliage, disease resistance, increased greenness of the crops providing a nutritionally rich crop.

Further, the various advantageous properties associated with the compositions according to the invention, include but are not limited to improved stability, improved toxicological and/or ecotoxicological behavior, improved crop characteristics including crop yields, crop qualities and characteristics and other advantages familiar to a person skilled in the art.

From the foregoing, it will be observed that numerous modifications and variations can be effectuated without departing from the true spirit and scope of the novel concepts of the present invention. It is to be understood that no limitation with respect to the specific embodiments illustrated is intended or should be inferred.

I claim:

1. A liquid suspension crop nutrition composition comprising a homogeneous mixture of:
at least one water insoluble Iron salt, complex or derivative thereof in the range of 1%-50% w/w of the total composition,
at least one water insoluble Zinc salt, complex or derivative thereof in the range of 1%-50% w/w of the total composition,
at least one water insoluble Magnesium salt, complex or derivative thereof in the range of 1%-70% w/w of the total composition,
at least one agrochemically acceptable excipient,
wherein elemental Iron is present in the range of 0.01% to 40% w/w of the total composition and
wherein elemental Zinc is present in the range of 0.01% to 40% w/w of the total composition and
wherein elemental Magnesium is present in the range of 0.01% to 50% w/w of the total composition and wherein the composition comprises particles in the size range of 0.1 micron-20 microns.

2. The liquid suspension crop nutrition composition as claimed in claim 1, wherein the composition is in the form of a suspension concentrate, oil dispersion or suspo-emulsion composition.

3. The liquid suspension composition as claimed in claim 1, wherein at least one water insoluble Iron salt comprises at least one of Iron Oxide, Iron Succinate, Iron Fumarate, Iron Hydroxide, Iron Oxalate, Iron Sucrate, Iron Tartrate, Iron Phosphate, Iron Carbonate, Iron Silicate, Carbonyl Iron, Iron Sulphide or Iron Dichromate, complex or derivative thereof.

4. The liquid suspension composition as claimed in claim 1, wherein at least one water insoluble Zinc salt comprises at least one of Zinc Oxide, Zinc Carbonate, Zinc Sulphide, Zinc Molybdate, Zinc Phosphate, Zinc Nitrilotriacetic Acid, Zinc Borate, Zinc Silicate, Zinc Pyrophosphate and Zinc Citrate, complex or derivative thereof.

5. The liquid suspension composition as claimed in claim 1, wherein at least one water insoluble Magnesium salt comprises at least one of Magnesium Molybdate, Magnesium Hydroxide, Calcium Magnesium Phosphate, Magnesium Carbonate, Magnesium Aluminium Silicate, Calcium Magnesium Silicate, Magnesium Trisilicate, Magnesium Silicate, Magnesium Oxide, complex or derivative thereof.

6. The liquid suspension composition as claimed in claim 1, wherein the composition comprises of particles in the size range of 0.1 micron to 10 microns.

7. The liquid suspension composition as claimed in claim 1, wherein the composition comprises particles having diameter distribution of D90 of about 15 microns.

8. The liquid suspension composition as claimed in claim 1, wherein the pourability of composition is less than 5% rinsed residue.

9. The liquid suspension composition as claimed in claim 1, wherein the viscosity of composition at 25° C. of about 10 cps to about 2000 cps.

10. The liquid suspension composition as claimed in claim 1, wherein suspensibility of the composition is at least 30%.

11. The liquid suspension composition as claimed in claim 1, wherein at least one agrochemically acceptable excipient is selected from one or more of wetting agents, surfactants, dispersing agents, emulsifiers, spreading agents, colorants, anticaking agents, structuring agents, binders, buffers or pH adjusters or neutralizing agents, tackifiers, pigments, stabilizers, antifoaming agents or defoamers, anti-settling agents, penetrants, solvents, preservatives.

12. The liquid suspension composition as claimed in claim 1 or 11, wherein the agrochemically acceptable excipient is in the range of 0.01%-97% w/w of the total composition.

13. The liquid suspension composition as claimed in claim 8, wherein the composition comprises at least one structuring agent.

14. The liquid suspension composition as claimed in claim 13, wherein the structuring agent is in the range of 0.01%-10% w/w of the total composition.

15. A process for preparation of liquid suspension composition as claimed in claim 1, wherein the process comprises:
a. milling a homogeneous blend of at least one water insoluble Iron salt, complex or derivative thereof in the range of 1%-50% w/w of the total composition; at least one water insoluble Zinc salt, complex or derivative thereof in the range of 1%-50% w/w of the total composition; at least one water insoluble Magnesium salt, complex or derivative thereof in the range of 1-70% w/w of the total composition, and at least one agrochemically acceptable excipient, in water or solvent to obtain a homogeneous slurry or wet mix with a particle size range of 0.1 micron to 20 microns, wherein elemental Iron is present in the range of 0.01% to 40% w/w of the total composition and
wherein elemental Zinc is present in the range of 0.01% to 40% w/w of the total composition and
wherein elemental Magnesium is present in the range of 0.01% to 50% w/w of the total composition.

16. A process of preparation of a liquid suspension composition in the form of oil dispersion as claimed in claim 1 comprises:
a. mixing of at least one water immiscible solvent, one or more surfactant to obtain a solvent-excipient mixture,
b. adding at least one water insoluble Iron salt, complex or derivative thereof in the range of 1%-50% w/w of the total composition; at least one water insoluble Zinc salt, complex or derivative thereof in the range of 1%-50% w/w of the total composition; at least one water insoluble Magnesium salt, complex or derivative thereof in the range of 1-70% w/w of the total composition and at least one agrochemical excipient to the solvent mixture to obtain a homogenized solution and
c. milling homogenized solution to obtain a dispersed medium of particle size in the range of 0.1 micron to 20 microns; wherein elemental Iron is present in the range of 0.01% to 40% w/w of the total composition and
wherein elemental Zinc is present in the range of 0.01% to 40% w/w of the total composition and
wherein elemental Magnesium is present in the range of 0.01% to 50% w/w of the total composition.

17. A process of preparation of a liquid suspension composition in the form of suspo-emulsion as claimed in claim 1 comprises:
a. mixing one or more of water insoluble Zinc salt, complex or derivative thereof, one or more of water insoluble Iron salt, complex or derivative thereof and one or more water insoluble Magnesium salt, complex or derivative thereof in an oil or a solvent and preparing a concentrated emulsion with required agrochemical excipients to obtain a first fraction.
b. mixing effective amount of surfactants or excipient to obtain a second fraction
c. mixing first and second fraction which is then milled to get the desired particle size of 0.1 to 50 microns.

18. A liquid suspension composition as claimed in claim 1, wherein the composition is at least one of a fertilizer composition, a nutrient composition, a crop strengthener composition, a soil conditioner composition and a yield enhancer composition.

19. A method for improving plant health or yield wherein the method comprises treating at least one of a plant, a plant propagation material, locus or parts thereof, a seed, seedling; or surrounding soil with the liquid suspension composition as claimed in claim 1.

20. A method for treating plants and meeting their nutritional requirement by enhancing uptake of Magnesium, Zinc and Iron by application of a liquid suspension composition comprising a homogeneous mixture of:
at least one water insoluble Iron salt, complex or derivative thereof in the range of 1%-50% w/w of the total composition,
at least one water insoluble Zinc salt, complex or derivative thereof in the range of 1%-50% w/w of the total composition,
at least one water insoluble Magnesium salt, complex or derivative thereof in the range of 1%-70% w/w of the total composition,
at least one agrochemically acceptable excipient,
wherein elemental Iron is present in the range of 0.01% to 40% w/w of the total composition and
wherein elemental Zinc is present in the range of 0.01% to 40% w/w of the total composition and
wherein elemental Magnesium is present in the range of 0.01% to 50% w/w of the total composition and
wherein the composition comprises particles in the size range of 0.1 micron-20 microns.
